(19) United States
(12) Patent Application Publication
(10) Pub. No.: US 2026/0157726 A1
(43) Pub. Date: Jun. 11, 2026

(54) Human Assisted Robotic Venipuncture Instrument

(71) Applicant: Takeda Pharmaceutical Company Limited, Osaka (JP)

(72) Inventors: Jacob Ward, Apex, NC (US); Ben Coble, Raleigh, NC (US); Joyce Minor, Raleigh, NC (US); Benjamin Kroll, Holly Springs, NC (US); Matthew D. Swecker, Raleigh, NC (US)

(73) Assignee: Takeda Pharmaceutical Company Limited, Osaka (JP)

(21) Appl. No.: 19/179,507

(22) Filed: Apr. 15, 2025

Related U.S. Application Data
(60) Provisional application No. 63/634,802, filed on Apr. 16, 2024.

(51) Int. Cl. A61B 8/00; A61B 8/08; A61B 17/34

(52) U.S. Cl. CPC A61B 8/4218; A61B 8/0891; A61B 8/429; A61B 8/5223; A61B 8/54; A61B 17/3403; A61B 2017/3413

(57) ABSTRACT

A method includes instructing an image capture device to move across an anatomy portion of a subject and capture a sequence of ultrasound image frames. For each corresponding ultrasound image frame, the method includes processing the corresponding ultrasound image frame to generate a respective vessel mask that identifies one or more vessel portions of the corresponding ultrasound image frame. The method also includes processing the vessel masks generated for the sequence of ultrasound image frames and corresponding three-dimensional position data to generate a three-dimensional vessel structure map representing vessels within the anatomy portion of the subject. The method also includes processing the three-dimensional vessel structure map to select a candidate vessel to target for venipuncture from the vessels represented in the three-dimensional vessel structure map.

### CROSS REFERENCE TO RELATED APPLICATIONS

This U.S. Patent Application claims priority under 35 U.S.C. § 119(e) to U.S. Provisional Application 63/634,802, filed on Apr. 16, 2024. The disclosure of this prior application is considered part of the disclosure of this application and is hereby incorporated by reference in its entirety.

### TECHNICAL FIELD

This disclosure relates to a human assisted robotic venipuncture instrument.

### BACKGROUND

Production of plasma derived therapies for humans requires the collection of plasma from human donors through plasmapheresis. In order to meet production goals, tens of millions of donations are required each year. Each donation requires a trained phlebotomist to perform venipuncture, therefore requiring thousands to be on staff at any given time. Average retention for phlebotomists can be as little as one year or less, resulting in a continuous stream of hiring and training personnel to perform venipuncture. Additionally, it takes several months for a phlebotomist to become proficient and often years to become an expert. The process also requires obtaining and retaining millions of willing donors with veins that are accessible by a human phlebotomist.

Veins under the skin are not visible in many people. A skilled phlebotomist relies more on touch or feel than on sight when determining if a vein is suitable for venipuncture. Palpation is used to assess the depth, width, direction and resilience of a vein. Even after palpation, many donor's veins are considered Difficult Venous Access (DVA) such that they are deferred from donation.

### SUMMARY

One aspect of the disclosure provides a computer-implemented method executed on data processing hardware that causes the data processing hardware to perform operations for site selection based on a sequence of ultrasound image frames. The operations include instructing an image capture device to move across an anatomy portion of a subject, and while the image capture device moves across the anatomy portion, capture a sequence of ultrasound image frames. For each corresponding ultrasound image frame in the sequence of ultrasound image frames, the operations also include processing, using a vessel identification model, the corresponding ultrasound image frame to generate a respective vessel mask that identifies one or more vessel portions of the corresponding ultrasound image frame. Each respective vessel portion indicates where a respective vessel is located in the corresponding ultrasound image frame. The operations further include, processing, using a vessel map generator, the vessel masks generated for the sequence of ultrasound image frames and corresponding three-dimensional position data to generate a three-dimensional vessel structure map representing vessels within the anatomy portion of the subject. Each respective vessel mask is paired with corresponding three-dimensional position data of the image capture device when the image capture device captured the corresponding ultrasound image frame. The operations also include processing the three-dimensional vessel structure map to select, from the vessels represented in the three-dimensional vessel structure map, a candidate vessel to target for venipuncture.

Implementations of the disclosure may include one or more of the following optional features. In some implementations, processing the three-dimensional vessel structure map to select the candidate vessel includes: processing the three-dimensional vessel structure map to identify a plurality of vessels within the anatomy portion of the subject; from each corresponding vessel of the plurality of vessels identified; extracting respective vessel properties of the corresponding vessel; ranking the plurality of vessels identified based on the respective vessel properties extracted for each of the plurality of vessels; and selecting the highest rank vessel among the plurality of vessels as the candidate vessel to target for venipuncture. In these implementations, the respective vessel properties extracted from each corresponding vessel may include at least one of: a diameter of the corresponding vessel, an angle of the corresponding vessel relative to a reference angle, a depth of the corresponding vessel from an exterior surface of the anatomy portion, or any branch vessels branching from the corresponding vessel. In some examples, the vessel identification model includes a deep neural network architecture.

In some implementations, for each corresponding ultrasound image frame in the sequence of ultrasound image frames, the operations further include: processing, using a contact detection model, the corresponding ultrasound image frame to generate a respective contact mask identifying the presence of any insufficient acoustic interface portions of the corresponding ultrasound image frame that indicate where an insufficient acoustic interface is located in the corresponding ultrasound image frame; comparing the respective vessel mask and the respective contact mask to determine whether the respective contact mask identified any insufficient acoustic interface portions that overlap with any of the vessel portions identified by the respective vessel mask in the corresponding ultrasound image frame; and validating the respective vessel mask to discard any vessel portions identified by the respective vessel mask that overlap with insufficient acoustic interface portions identified by the respective contact mask. Here, processing the vessel masks generated for the sequence of ultrasound image frames may include processing, using the vessel map generator, the validated vessel masks and the corresponding three-dimensional position data to generate the three-dimensional vessel structure map. In these implementations, the insufficient acoustic interface may indicate an insufficient acoustic interface between an ultrasound sensor of the image capture device and the anatomy portion of the subject. In these implementations, the vessel identification model may include a first deep neural network architecture configured to receive, as input, the sequence of ultrasound image frames and generate, as output, the vessel masks; and the contact detection model may include a second deep neural network architecture different from the first neural network and configured to receive, as input, the sequence of ultrasound image frames and generate, as output, the contact masks. Alternatively, the vessel identification model and the contact detection model may each include a same deep neural network architecture configured to receive, as input, the sequence of ultrasound image frames and generate, as output, both the vessel masks and the contact masks.

Another aspect of the disclosure provides a computer-implemented method executed on data processing hardware that causes the data processing hardware to perform operations for vein confirmation based on a sequence of ultrasound image frames. The operations include receiving a three-dimensional vessel structure map representing vessels of an anatomy portion of a subject in a three-dimensional space. The operations further include processing the three-dimensional vessel structure map to select: a candidate vessel from the vessels represented in the three-dimensional vessel structure map to target for venipuncture; and an initial target location of the selected candidate vessel to puncture. The operations also include instructing an ultrasound image device to: move to a target position against the anatomy portion of the subject based on the initial target location of the candidate vessel; apply, from the target position against the anatomy portion of the subject, pressure against the anatomy portion to exert a force upon the candidate vessel at the initial target location; and capture a sequence of ultrasound image frames while the ultrasound image devices is applying the pressure against the anatomy portion of the subject from the target position. The operations further include processing the sequence of ultrasound image frames captured by the ultrasound image device to extract compressive properties of the candidate vessel, determining the candidate vessel includes a vein based on the compressive properties of the candidate vessel, and based on determining the candidate vessel includes the vein, instructing a cannula positioning device to insert a cannula into the candidate vessel that includes the vein.

Implementations of the disclosure may include one or more of the following optional features. In some implementations, instructing the ultrasound image device to move to the target position further includes instructing the ultrasound image device to move to a target orientation that aligns a longitudinal axis of the ultrasound image in a direction substantially perpendicular to a longitudinal axis of the candidate vessel at the target location. Here, instructing the ultrasound image device to apply pressure includes instructing the ultrasound image device to apply, from the target position and the target orientation, the pressure against the anatomy portion to exert the force upon the candidate vessel in the direction substantially perpendicular to the longitudinal axis of the candidate vessel at the target location. In some examples, instructing the ultrasound image device to apply pressure includes instructing the ultrasound image device to increase pressure from an initial pressure value to a final pressure value during a predetermined duration of time.

In some implementations, determining the candidate vessel includes a vein includes executing a vein confirmation model configured to: receive, as input, the compressive properties of the candidate vessel and a magnitude of the force exerted upon the candidate vessel at the target location; and generate a classification output classifying the candidate vessel as the vein. In these implementations, the vein confirmation model may be trained to: classify vessels as a vein when the compressive properties of the vessels indicate a decreasing cross-sectional area responsive to increases in magnitude of force exerted upon the vessels; and classify vessels as arteries when the compressive properties of the vessels indicate that the cross-sectional areas does not decrease responsive to increases in the magnitude of force.

In some examples, the operations further include, based on determining that the candidate vessel includes the vein, instructing the cannula positioning device to orient a longitudinal axis of the cannula at a target angle relative to a longitudinal axis of the vein. Here, instructing the cannula positioning device to insert the cannula into the candidate vessel that includes the vein includes instructing the cannula positioning device to insert the cannula into the candidate vessel while the longitudinal axis of the cannula is oriented at the target angle relative to the longitudinal axis of the vein. In some implementations, processing the three-dimensional vessel structure map to select the candidate vessel includes: processing the three-dimensional vessel structure map to identify a plurality of vessels within the anatomy portion of the subject; from each corresponding vessel of the plurality of vessels identified, extracting respective vessel properties of the corresponding vessel; ranking the plurality of vessels identified based on the respective vessel properties extracted for each of the plurality of vessels; and selecting the highest rank vessel among the plurality of vessels as the candidate vessel to target for venipuncture. In these implementations, the respective vessel properties extracted from each corresponding vessel may include at least one of: a diameter of the corresponding vessel, an angle of the corresponding vessel relative to a reference angle, a depth of the corresponding vessel from an exterior surface of the anatomy portion, or any branch vessels branching from the corresponding vessel.

In some examples, processing the sequence of ultrasound image frames captured by the ultrasound image device to extract compressive properties of the candidate vessel includes, for each ultrasound image frame in the sequence of ultrasound image frames: processing, using a vessel identification model, the corresponding ultrasound image frame to generate a respective vessel mask that identifies a respective portion of the corresponding ultrasound image frame where the candidate vessel is located; and processing the respective vessel mask to determine a cross-sectional area of the candidate vessel. Additionally, processing the sequence of ultrasound image frames captured by the ultrasound image device to extract compressive properties of the candidate vessel further includes determining the compressive properties of the candidate vessel based on the cross-sectional areas of the candidate vessel determined for the sequence of ultrasound image frames.

In some implementations, the sequence of ultrasound image frames include two-dimensional ultrasound image frames. In some examples, the operations further include, after determining the candidate vessel includes the vein: instructing the image capture device to capture, from the target position against the anatomy portion of the subject, an additional ultrasound image frame; and processing the additional ultrasound image frame to identify the candidate vessel and determine a final target location of the candidate vessel to puncture. Here, instructing the cannula positioning device to insert the cannula into the candidate vessel includes instructing the cannula positioning device to insert the cannula into the candidate vessel at the final target location.

Another aspect of the disclosure provides a computer-implemented method executed on data processing hardware that causes the data processing hardware to perform operations for training a vessel identification model and a contact detection model. The operations include receiving a training corpus of ultrasound image sequence sets with each ultrasound image sequence set including a corresponding sequence of ultrasound image frames of the anatomy portion captured by a corresponding ultrasound image device as the corresponding ultrasound image device scans across the anatomy portion. Here, each corresponding ultrasound image frame includes manual annotations that identify one or more corresponding ground-truth vessel locations in the corresponding ultrasound image frame and is paired with three-dimensional positional data of the corresponding ultrasound image device when the corresponding ultrasound image frame was captured by the corresponding ultrasound image device. For each ultrasound image sequence set in the training corpus, the operations further include training a vessel identification model on the corresponding sequence of ultrasound image frames to teach the vessel identification model to learn how to generate a corresponding predicted vessel mask for each corresponding ultrasound image frame that identifies the one or more corresponding ground-truth vessel locations.

In some implementations, the vessel identification model includes a deep neural network. In these implementations, training the vessel identification model on the corresponding sequence of ultrasound image frames may include: for each corresponding ultrasound image frame in the corresponding sequence of ultrasound image frames, processing the ultrasound image frame to generate one or more predicted vessel masks using the deep neural network and determining a loss term based on the one or more predicted vessel masks and the manual annotations that identify the one or more corresponding ground-truth vessel locations in the corresponding ultrasound image frame; and updating parameters of the deep neural network based on the loss terms determined for the corresponding sequence of ultrasound image frames.

In some examples, for each respective ultrasound image frame from the training corpus of ultrasound image sequence sets that includes the presence of an insufficient acoustic interface, the respective ultrasound image frame further includes additional manual annotations that identify one or more corresponding ground-truth insufficient acoustic interface locations in the respective ultrasound image frame. Here, the operations further include, for each respective ultrasound image frame from the training corpus of ultrasound image sequence sets that includes the presence of the insufficient acoustic interface, training a contact detection model on each respective ultrasound image frame to teach the contact detection model to learn how to generate a corresponding predicted contact detection mask for each respective ultrasound image frame that identifies the one or more corresponding ground-truth insufficient acoustic interface locations. In these examples, the vessel identification model may include a first deep neural network architecture and the contact detection model may include a second deep neural network architecture different from the first neural network. Alternatively, the vessel identification model and the contact detection model may each include a same deep neural network architecture.

In some implementations, for each ultrasound image sequence set in the training corpus, the operations further include, processing, using a vessel map generator, the one or more corresponding ground-truth vessel locations identified in each corresponding ultrasound image frame and the three-dimensional positional data paired with each corresponding ultrasound image frame to generate a corresponding three-dimensional vessel structure map representing vessels of the anatomy portion in a three-dimensional space. In these implementations, the corresponding three-dimensional structure map may be labeled to identify a ground-truth target vessel from the vessels represented in the three-dimensional vessel structure map to target for venipuncture and a ground-truth target location of the ground-truth target vessel to puncture. Here, the operations further include training a venipuncture site selection model on the corresponding three-dimensional structure maps to teach the venipuncture site selection model to learn how to predict target vessels to target for venipuncture and target locations of the predicted target vessels to puncture. In some examples, each corresponding ultrasound image frame includes a two-dimensional ultrasound image frame.

Another aspect of the disclosure provides a venipuncture device including data processing hardware and memory hardware in communication with the data processing hardware. The memory hardware stores instructions that when executed on the data processing hardware cause the data processing hardware to perform operations. The operations include instructing an image capture device to move across an anatomy portion of a subject, and while the image capture device moves across the anatomy portion, capture a sequence of ultrasound image frames. For each corresponding ultrasound image frame in the sequence of ultrasound image frames, the operations also include processing, using a vessel identification model, the corresponding ultrasound image frame to generate a respective vessel mask that identifies one or more vessel portions of the corresponding ultrasound image frame. Each respective vessel portion indicates where a respective vessel is located in the corresponding ultrasound image frame. The operations further include, processing, using a vessel map generator, the vessel masks generated for the sequence of ultrasound image frames and the corresponding three-dimensional position data to generate a three-dimensional vessel structure map representing vessels within the anatomy portion of the subject. Each respective vessel mask is paired with corresponding three-dimensional position data of the image capture device when the image capture device captured the corresponding ultrasound image frame. The operations also include processing the three-dimensional vessel structure map to select, from the vessels represented in the three-dimensional vessel structure map, a candidate vessel to target for venipuncture.

Implementations of the disclosure may include one or more of the following optional features. In some implementations, processing the three-dimensional vessel structure map to select the candidate vessel includes: processing the three-dimensional vessel structure map to identify a plurality of vessels within the anatomy portion of the subject; from each corresponding vessel of the plurality of vessels identified; extracting respective vessel properties of the corresponding vessel; ranking the plurality of vessels identified based on the respective vessel properties extracted for each of the plurality of vessels; and selecting the highest rank vessel among the plurality of vessels as the candidate vessel to target for venipuncture. In these implementations, the respective vessel properties extracted from each corresponding vessel may include at least one of: a diameter of the corresponding vessel, an angle of the corresponding vessel relative to a reference angle, a depth of the corresponding vessel from an exterior surface of the anatomy portion, or any branch vessels branching from the corresponding vessel. In some examples, the vessel identification model includes a deep neural network architecture.

In some implementations, for each corresponding ultrasound image frame in the sequence of ultrasound image frames, the operations further include: processing, using a contact detection model, the corresponding ultrasound image frame to generate a respective contact mask identifying the presence of any insufficient acoustic interface portions of the corresponding ultrasound image frame that indicate where an insufficient acoustic interface is located in the corresponding ultrasound image frame; comparing the respective vessel mask and the respective contact mask to determine whether the respective contact mask identified any insufficient acoustic interface portions that overlap with any of the vessel portions identified by the respective vessel mask in the corresponding ultrasound image frame; and validating the respective vessel mask to discard any vessel portions identified by the respective vessel mask that overlap with insufficient acoustic interface portions identified by the respective contact mask. Here, processing the vessel masks generated for the sequence of ultrasound image frames may include processing, using the vessel map generator, the validated vessel masks and the corresponding three-dimensional position data to generate the three-dimensional vessel structure map. In these implementations, the insufficient acoustic interface may indicate an insufficient acoustic interface between an ultrasound sensor of the image capture device and the anatomy portion of the subject. In these implementations, the vessel identification model may include a first deep neural network architecture configured to receive, as input, the sequence of ultrasound image frames and generate, as output, the vessel masks; and the contact detection model may include a second deep neural network architecture different from the first neural network and configured to receive, as input, the sequence of ultrasound image frames and generate, as output, the contact masks. Alternatively, the vessel identification model and the contact detection model may each include a same deep neural network architecture configured to receive, as input, the sequence of ultrasound image frames and generate, as output, both the vessel masks and the contact masks.

Another aspect of the disclosure provides a venipuncture device including data processing hardware and memory hardware in communication with the data processing hardware. The memory hardware stores instructions that when executed on the data processing hardware cause the data processing hardware to perform operations. The operations include receiving a three-dimensional vessel structure map representing vessels of an anatomy portion of a subject in a three-dimensional space. The operations further include processing the three-dimensional vessel structure map to select: a candidate vessel from the vessels represented in the three-dimensional vessel structure map to target for venipuncture; and an initial target location of the selected candidate vessel to puncture. The operations also include instructing an ultrasound image device to: move to a target position against the anatomy portion of the subject based on the initial target location of the candidate vessel; apply, from the target position against the anatomy portion of the subject, pressure against the anatomy portion to exert a force upon the candidate vessel at the initial target location; and capture a sequence of ultrasound image frames while the ultrasound image devices is applying the pressure against the anatomy portion of the subject from the target position. The operations further include processing the sequence of ultrasound image frames captured by the ultrasound image device to extract compressive properties of the candidate vessel, determining the candidate vessel includes a vein based on the compressive properties of the candidate vessel, and based on determining the candidate vessel includes the vein, instructing a cannula positioning device to insert a cannula into the candidate vessel that includes the vein.

Implementations of the disclosure may include one or more of the following optional features. In some implementations, instructing the ultrasound image device to move to the target position further includes instructing the ultrasound image device to move to a target orientation that aligns a longitudinal axis of the ultrasound image in a direction substantially perpendicular to a longitudinal axis of the candidate vessel at the target location. Here, instructing the ultrasound image device to apply pressure includes instructing the ultrasound image device to apply, from the target position and the target orientation, the pressure against the anatomy portion to exert the force upon the candidate vessel in the direction substantially perpendicular to the longitudinal axis of the candidate vessel at the target location. In some examples, instructing the ultrasound image device to apply pressure includes instructing the ultrasound image device to increase pressure from an initial pressure value to a final pressure value during a predetermined duration of time.

In some implementations, determining the candidate vessel includes a vein includes executing a vein confirmation model configured to: receive, as input, the compressive properties of the candidate vessel and a magnitude of the force exerted upon the candidate vessel at the target location; and generate a classification output classifying the candidate vessel as the vein. In these implementations, the vein confirmation model may be trained to: classify vessels as a vein when the compressive properties of the vessels indicate a decreasing cross-sectional area responsive to increases in magnitude of force exerted upon the vessels; and classify vessels as arteries when the compressive properties of the vessels indicate that the cross-sectional areas does not decrease responsive to increases in the magnitude of force.

In some examples, the operations further include, based on determining the candidate vessel includes the vein, instructing the cannula positioning device to orient a longitudinal axis of the cannula at a target angle relative to a longitudinal axis of the vein. Here, instructing the cannula positioning device to insert the cannula into the candidate vessel that includes the vein includes instructing the cannula positioning device to insert the cannula into the candidate vessel while the longitudinal axis of the cannula is oriented at the target angle relative to the longitudinal axis of the vein. In some implementations, processing the three-dimensional vessel structure map to select the candidate vessel includes: processing the three-dimensional vessel structure map to identify a plurality of vessels within the anatomy portion of the subject; from each corresponding vessel of the plurality of vessels identified, extracting respective vessel properties of the corresponding vessel; ranking the plurality of vessels identified based on the respective vessel properties extracted for each of the plurality of vessels; and selecting the highest rank vessel among the plurality of vessels as the candidate vessel to target for venipuncture. In these implementations, the respective vessel properties extracted from each corresponding vessel may include at least one of: a diameter of the corresponding vessel, an angle of the corresponding vessel relative to a reference angle, a depth of the corresponding vessel from an exterior surface of the anatomy portion, or any branch vessels branching from the corresponding vessel.

In some examples, processing the sequence of ultrasound image frames captured by the ultrasound image device to extract compressive properties of the candidate vessel includes, for each ultrasound image frame in the sequence of ultrasound image frames: processing, using a vessel identification model, the corresponding ultrasound image frame to generate a respective vessel mask that identifies a respective portion of the corresponding ultrasound image frame where the candidate vessel is located; and processing the respective vessel mask to determine a cross-sectional area of the candidate vessel. Additionally, processing the sequence of ultrasound image frames captured by the ultrasound image device to extract compressive properties of the candidate vessel further includes determining the compressive properties of the candidate vessel based on the cross-sectional areas of the candidate vessel determined for the sequence of ultrasound image frames.

In some implementations, the sequence of ultrasound image frames include two-dimensional ultrasound image frames. In some examples, the operations further include, after determining the candidate vessel includes the vein: instructing the image capture device to capture, from the target position against the anatomy portion of the subject, an additional ultrasound image frame; and processing the additional ultrasound image frame to identify the candidate vessel and determine a final target location of the candidate vessel to puncture. Here, instructing the cannula positioning device to insert the cannula into the candidate vessel includes instructing the cannula positioning device to insert the cannula into the candidate vessel at the final target location.

Another aspect of the disclosure provides a system that includes data processing hardware and memory hardware in communication with the data processing hardware. The memory hardware stores instructions that when executed on the data processing hardware cause the data processing hardware to perform operations. The operations include receiving a training corpus of ultrasound image sequence sets with each ultrasound image sequence set including a corresponding sequence of ultrasound image frames of the anatomy portion captured by a corresponding ultrasound image device as the corresponding ultrasound image device scans across the anatomy portion. Here, each corresponding ultrasound image frame: includes manual annotations that identify one or more corresponding ground-truth vessel locations in the corresponding ultrasound image frame; and is paired with three-dimensional positional data of the corresponding ultrasound image device when the corresponding ultrasound image frame was captured by the corresponding ultrasound image device. For each ultrasound image sequence set in the training corpus, the operations further include training a vessel identification model on the corresponding sequence of ultrasound image frames to teach the vessel identification model to learn how to generate a corresponding predicted vessel mask for each corresponding ultrasound image frame that identifies the one or more corresponding ground-truth vessel locations.

In some implementations, the vessel identification model includes a deep neural network. In these implementations, training the vessel identification model on the corresponding sequence of ultrasound image frames may include: for each corresponding ultrasound image frame in the corresponding sequence of ultrasound image frames, processing, using the deep neural network, the ultrasound image frame to generate one or more predicted vessel masks and determining a loss term based on the one or more predicted vessel masks and the manual annotations that identify the one or more corresponding ground-truth vessel locations in the corresponding ultrasound image frame; and updating parameters of the deep neural network based on the loss terms determined for the corresponding sequence of ultrasound image frames.

In some examples, for each respective ultrasound image frame from the training corpus of ultrasound image sequence sets that includes the presence of an insufficient acoustic interface, the respective ultrasound image frame further includes additional manual annotations that identify one or more corresponding ground-truth insufficient acoustic interface locations in the respective ultrasound image frame. Here, the operations further include, for each respective ultrasound image frame from the training corpus of ultrasound image sequence sets that includes the presence of the insufficient acoustic interface, training a contact detection model on each respective ultrasound image frame to teach the contact detection model to learn how to generate a corresponding predicted contact detection mask for each respective ultrasound image frame that identifies the one or more corresponding ground-truth insufficient acoustic interface locations. In these examples, the vessel identification model may include a first deep neural network architecture and the contact detection model may include a second deep neural network architecture different from the first neural network. Alternatively, the vessel identification model and the contact detection model each may include a same deep neural network architecture.

In some implementations, for each ultrasound image sequence set in the training corpus, the operations further include, processing, using a vessel map generator, the one or more corresponding ground-truth vessel locations identified in each corresponding ultrasound image frame and the three-dimensional positional data paired with each corresponding ultrasound image frame to generate a corresponding three-dimensional vessel structure map representing vessels of the anatomy portion in a three-dimensional space. In these implementations, the corresponding three-dimensional structure map may be labeled to identify: a ground-truth target vessel from the vessels represented in the three-dimensional vessel structure map to target for venipuncture; and a ground-truth target location of the ground-truth target vessel to puncture; and the operations may further include training a venipuncture site selection model on the corresponding three-dimensional structure maps to teach the venipuncture site selection model to learn how to predict target vessels to target for venipuncture and target locations of the predicted target vessels to puncture. In some examples, each corresponding ultrasound image frame includes a two-dimensional ultrasound image frame.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1A is a side view of an example venipuncture device.

FIG. 1B is a schematic view of an example venipuncture device.

FIG. 1C is a schematic view of a sensor arrangement of two optical sensors of the example venipuncture device.

FIGS. 2A-2H are schematic views of multiple degrees of freedom of the example venipuncture device of FIG. 1A.

FIG. 3 is a schematic view of an example site selection process executed by the venipuncture device.

FIG. 4 is a schematic view of an example vessel confirmation process executed by the venipuncture device.

FIG. 5 is a graphical view of a first example ultrasound image frame input to a vessel identification model and a corresponding vessel mask output by the vessel identification model.

FIG. 6 is a graphical view of a second example ultrasound image frame input to a contact detection model and a corresponding contact mask output by the contact model.

FIG. 7 is a graphical view of an example three-dimensional vessel structure map output from a validation module.

FIG. 8 is a graphical view of an example three-dimensional site selection map including a candidate vessel and an initial target location output from a site selector.

FIG. 9 depicts a sequence of images representing the venipuncture device performing the site selection process of FIG. 3.

FIG. 10 depicts a sequence of images representing the venipuncture device performing the vessel confirmation process of FIG. 4.

FIG. 11A is a schematic view of an example vessel identification model training process.

FIG. 11B is a schematic view of an example contact detection model training process.

FIG. 11C is a schematic view of an example joint training process for the vessel identification model and the contact detection model training process.

FIG. 12 is a flowchart of an example arrangement of operations for a computer-implemented method of performing a site selection process.

FIG. 13 is a flowchart of an example arrangement of operations for a computer-implemented method of performing a vessel confirmation process.

FIG. 14 is a flowchart of an example arrangement of operations for a computer-implemented method of training a vessel identification model.

FIG. 15 is a schematic view of an example computing device that may be used to implement the systems and methods described herein.

Like reference symbols in the various drawings indicate like elements.

### DETAILED DESCRIPTION

Production of plasma derived therapies for humans requires the collection of plasma from human donors through plasmapheresis. To that end, human donors undergo a venipuncture procedure whereby a cannula punctures a vein of the donor typically to withdraw blood or for an intravenous injection. Conventionally, venipuncture requires a trained phlebotomist to perform the procedure. However, the number of trained phlebotomists is often insufficient for the demand of venipuncture procedures. Moreover, a significant amount of variation occurs in the venipuncture procedure depending on the training and skill level of the phlebotomists.

Accordingly, implementations herein are directed toward a venipuncture device and method for performing a site selection process to select a candidate vessel for venipuncture. That is, the site selection process instructs an image capture device to move across an anatomy portion of a subject and to capture a sequence of ultrasound image frames. The site selection process uses a vessel identification model to process each corresponding ultrasound image frame to generate a respective vessel mask that identifies one or more vessel portions of the corresponding ultrasound image frame. The site selection process uses a vessel map generator to process the vessel masks and corresponding three-dimensional position data to generate a three-dimensional vessel structure map representing vessels within the anatomy portion of the subject. Each respective vessel mask is paired with corresponding three-dimensional position data of the image capture device when the image capture device captured the corresponding ultrasound image frame. Thereafter, the site selection process selects a candidate vessel to target for venipuncture from a plurality of vessels represented in the three-dimensional vessel structure map. However, in some scenarios, the candidate vessel is an artery (not a vein), and thus, is not suitable for venipuncture. Moreover, the subject may have moved from the time the image capture device captured the ultrasound image frames, such that an initial target location is no longer aligned with the candidate vessel.

To that end, implementations herein are further directed towards a venipuncture device and method for performing a vein confirmation process. Here, the vein confirmation process receives a three-dimensional vessel structure map representing vessels of an anatomy portion of a subject in a three-dimensional space and processes the three-dimensional vessel structure map to select a candidate vessel from the vessels represented in the three-dimensional vessel structure map to target for venipuncture and to select an initial target location of the selected candidate vessel to puncture. Thereafter, the vein confirmation process instructs an image capture device to move to a target position (e.g., a position where the image capture device was located when the image capture device captured the respective ultrasound image that includes the candidate vessel), to apply pressure against the anatomy portion from the target position, and to capture a sequence of ultrasound image frames while the image capture device applies pressure against the anatomy portion. The vein confirmation process processes the sequence of ultrasound image frames captured by the image capture device while the image capture device applies pressure from the target position and determines whether the candidate vessel is a vein or artery. Based on determining that the candidate vessel is a vein, the vein confirmation process instructs a cannula positioning device to insert a cannula into the candidate vessel that includes the vein. However, as will become apparent, the vein confirmation process may perform additional steps, such as site confirmation, before instructing the cannula positioning device to insert the cannula into the candidate vessel that includes the vein.

Implementations herein are further directed towards a method and system for training a vessel identification model. In particular, a training process receives a training corpus of ultrasound image sequence sets where each set includes a corresponding sequence of ultrasound image frames of the anatomy portion captured by a corresponding ultrasound image device as the corresponding ultrasound image device scans across the anatomy portion. Here, each ultrasound image frame includes manual annotations that identify one or more corresponding ground-truth vessel locations in the corresponding ultrasound image frame and may be paired with three-dimensional positional data of the corresponding ultrasound image device when the corresponding ultrasound image frame was captured by the corresponding ultrasound image device. In some examples, the training process does not require knowledge of the three-dimensional positional data when each ultrasound image frame was captured because the vessel identification model operates on a single ultrasound image frame at a time and does not need knowledge of the image position. For each ultrasound image sequence set, the training process trains the vessel identification model on the corresponding sequence of ultrasound image frames to teach the vessel identification model how to generate a corresponding predicted vessel mask for each corresponding ultrasound image frame that identifies the one or more corresponding ground-truth vessel locations.

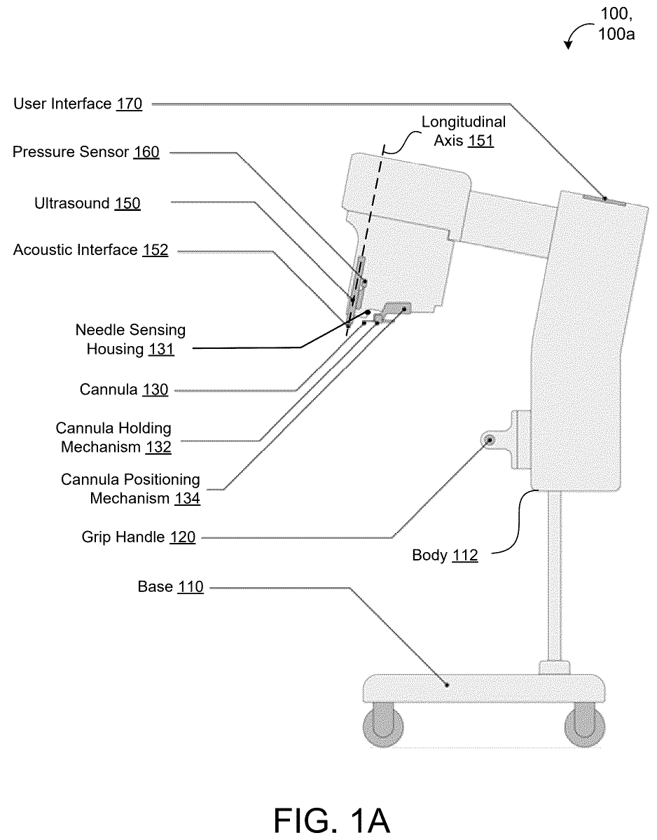

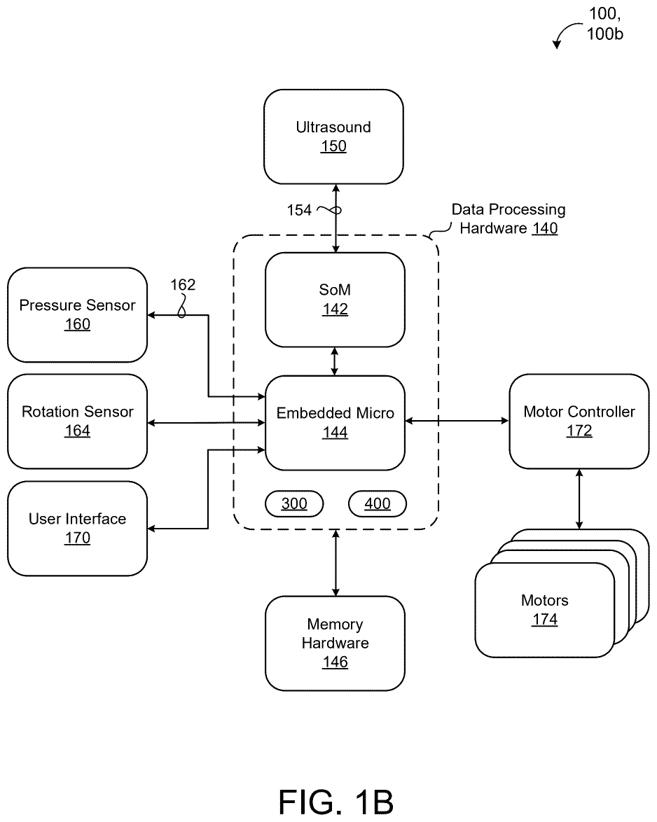

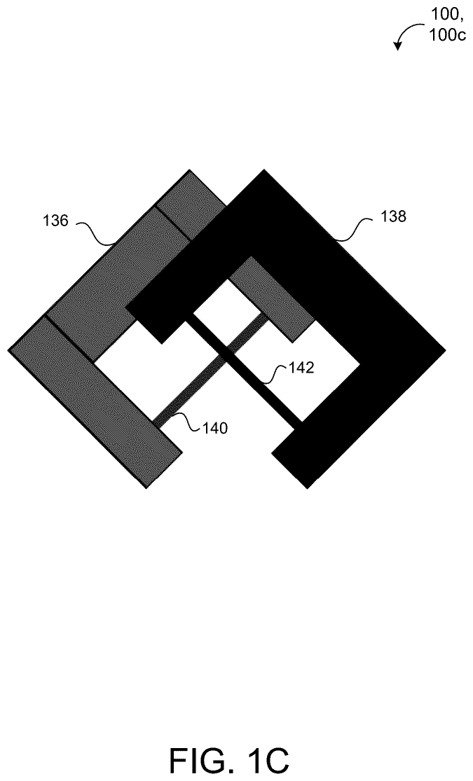

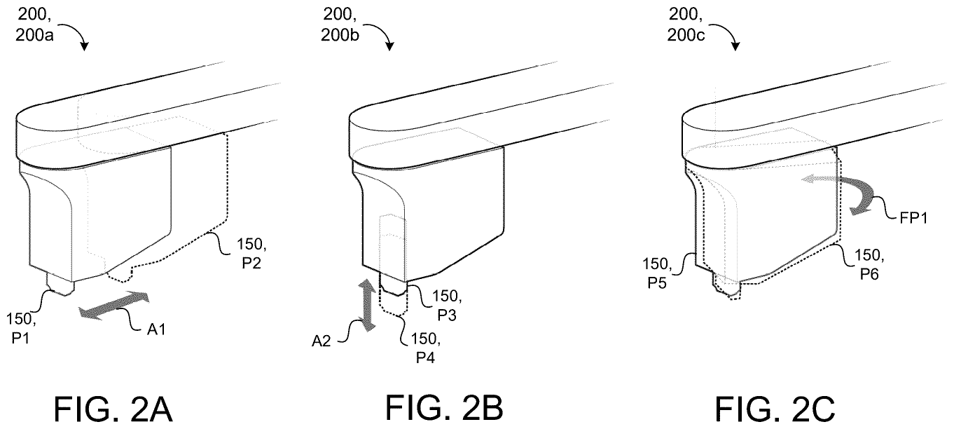

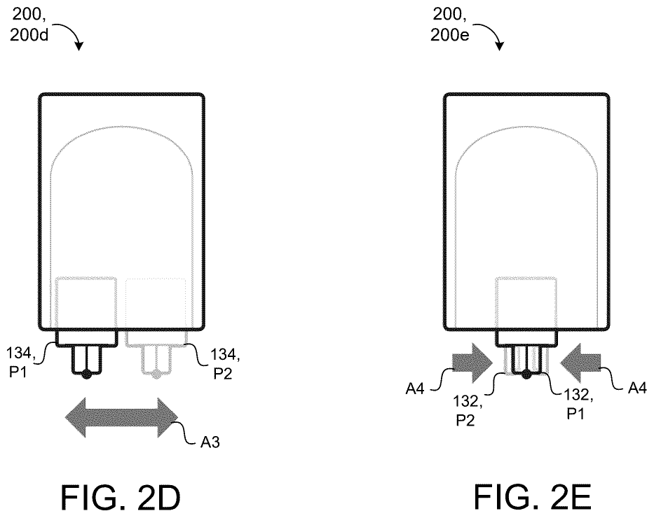

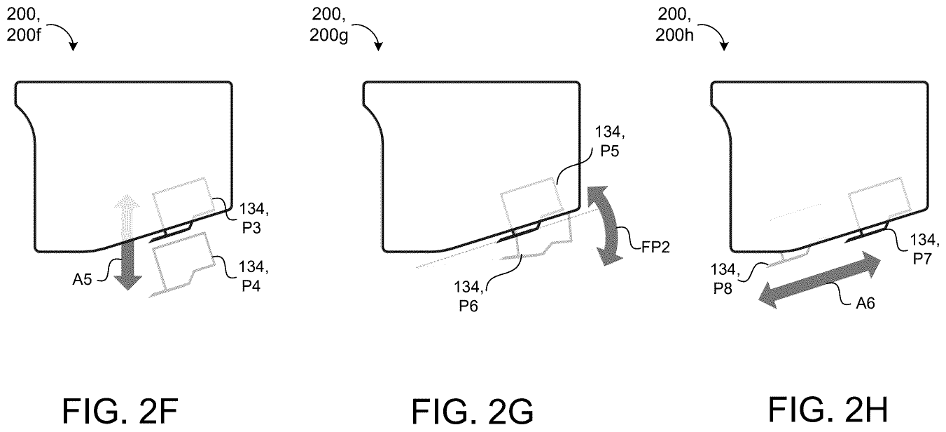

FIGS. 1A and 1B illustrate an example venipuncture device 100. In particular, FIG. 1A illustrates a side view 100, 100a of the example venipuncture device 100. In some implementations, the venipuncture device 100 includes a base 110 attached to a body 112. Here, the base 110 may be a movable base that allows a patient or operator of the venipuncture device 100 to move the image capture device 150 within an environment. The venipuncture device 100 may also include a grip handle 120 disposed on the body 112 whereby the patient (i.e., subject) grasps the grip handle 120 during the venipuncture procedure. The venipuncture device 100 also includes a cannula 130, a cannula holding mechanism 132, and a cannula positioning mechanism 134. Here, the cannula positioning mechanism 134 may be attached to the body 112 of the venipuncture device 100 and the cannula holding mechanism 132 is attached to the cannula positioning mechanism 134. Moreover, the cannula holding mechanism 132 secures the cannula 130 that is inserted in an anatomy portion of the subject during the venipuncture procedure. The venipuncture device 100 may also include a needle sensing housing 131 to house a verification station or sensor arrangement for performing a needle verification process, as described in FIG. 1C. Described in greater detail with reference to FIG. 2, the cannula positioning mechanism 134 is operable to position the cannula holding mechanism 132 and the cannula positioning mechanism 134 to a target position.

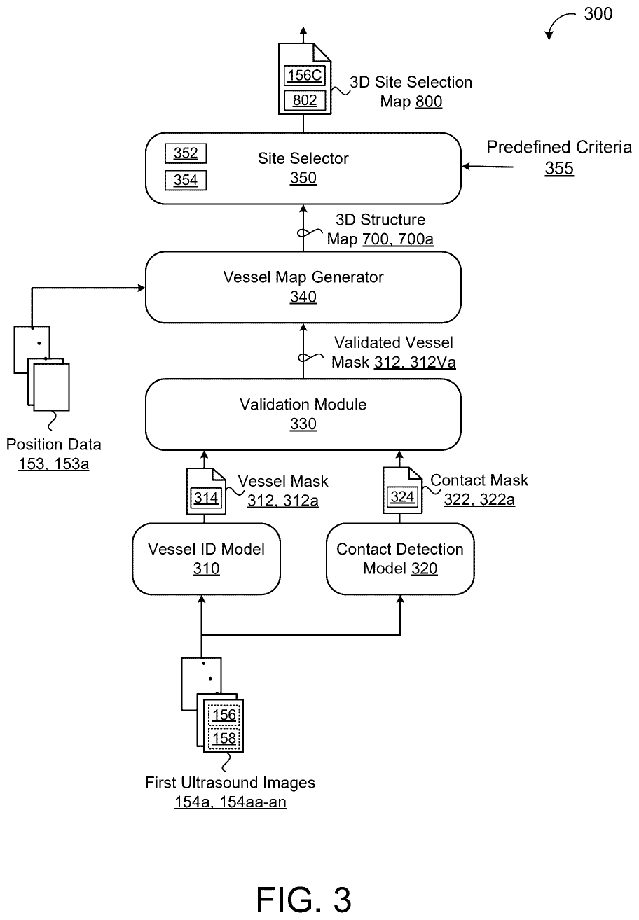

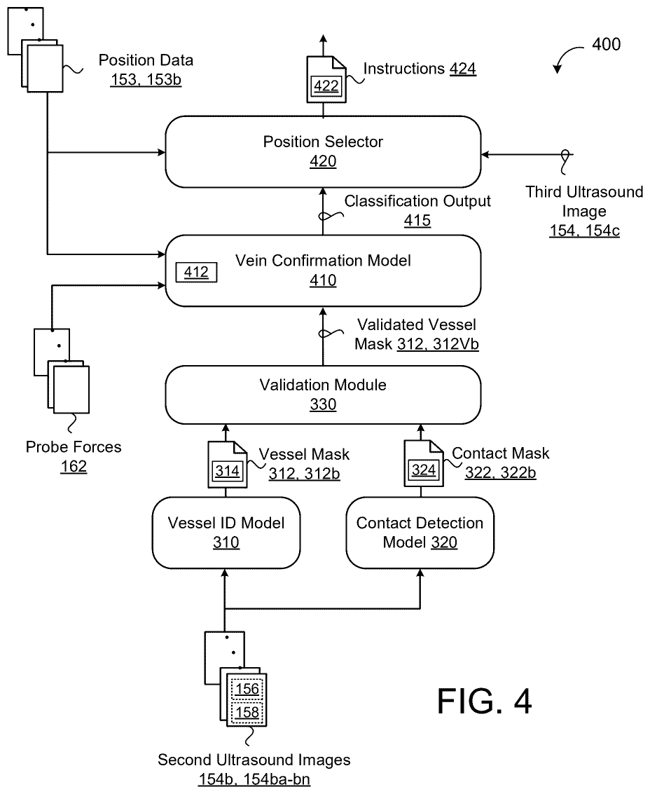

In some examples, the venipuncture device 100 includes an ultrasonic device 150 as the image capture device 150. As such, the image capture device 150 may interchangeably be referred to as the ultrasonic device 150 herein. The ultrasonic device 150 may include an ultrasound imaging probe. In these examples, the ultrasonic device 150 has an acoustic interface 152 and a pressure sensor 160. A force sensor may be implemented in addition to, or in lieu of, the pressure sensor 160. The acoustic interface 152 may include a gel clip that contacts the anatomy portion of the subject to enable the ultrasonic device 150 to capture ultrasound image frames 154 (FIG. 3) as the ultrasonic device 150 moves across an anatomy portion of the subject (e.g., the cubital area of a patient's arm). Alternatively, the image capture device 150 captures image frames 154 as the image capture device 150 moves across the anatomy portion of the subject. Thus, ultrasound image frames 154 and images frames 154 may be used interchangeably herein. The ultrasonic device 150 defines a longitudinal axis 151 extending along a length of the ultrasonic device 150. Moreover, the pressure sensor 160 may capture probe forces 162 (FIG. 4) as the ultrasonic device 150 moves across an anatomy portion of the subject. The cannula 130, the cannula holding mechanism 132, the cannula positioning mechanism 134, the ultrasonic device 150, and the acoustic interface 152 may collectively be referred to as a venipuncture arm 200 (FIG. 2). The venipuncture device 100 may include a user interface 170 (e.g., a graphical user interface (GUI)) that the operator of the venipuncture device 100 may interact with (e.g., via user input interactions) to operate the venipuncture device 100. For instance, the operator may provide touch inputs to interact with the user interface 170.

FIG. 1B illustrates a schematic view 100, 100b of the example venipuncture device 100 that includes data processing hardware 140. The data processing hardware 140 may reside locally at the venipuncture device 100 and/or at a remote computing system (e.g., distributed computing system such as a cloud computing environment). The data processing hardware 140 may include a system on module (SoM) component 142 and an embedded microprocessor 144 in communication with the SoM component 142. The SoM component 142 is also in communication with the ultrasonic device 150 such that the SoM component 142 receives ultrasound image frames 154 captured by the ultrasonic device 150. The ultrasound image frames 154 may include two-dimensional image frames. In some implementations, the embedded microprocessor 144 is in communication with a set of sensors to aid in operation of the venipuncture device 100. For instance, the embedded microprocessor 144 is in communication with the pressure sensor (i.e., force sensor) 160, a rotation sensor 164, and the user interface 170. In particular, the embedded microprocessor 144 receives probe forces 162 captured by the pressure sensor 160 and rotational data (i.e., pose data) of the venipuncture device 100 from the rotation sensor 164.

In some examples, the embedded microprocessor 144 is in communication with a motor controller 172 that instructs one or more motors 174 of the venipuncture device 100. For instance, the motor controller 172 may instruct the one or more motors 174 to position the ultrasonic device 150 and/or cannula 130 (e.g., via the cannula positioning mechanism 134). While the example shown depicts the motor controller 172 separate from the data processing hardware 140, it is understood that, in other examples, the motor controller 172 may be integrated with the data processing hardware 140 (not shown). The data processing hardware 140 is also in communication with memory hardware 146 that stores instructions that when executed on the data processing hardware 140 causes the data processing hardware 140 to perform operations. For instance, described in greater detail below with reference to FIGS. 3 and 4, the data processing hardware 140 may perform operations to execute a site selection process 300 (FIG. 3) and/or a vein confirmation process 400 (FIG. 4).

FIGS. 2A-2H illustrate multiple degrees of freedom 200 of the venipuncture device 100. That is, the venipuncture device 100 may include a robotic arm configured to position the cannula 130 and/or the ultrasonic device 150 at a target position against the anatomy portion of a subject. For instance, FIG. 2A illustrates a first degree of freedom (DOF) 200, 200a of the venipuncture device 100 that enables the ultrasonic device 150 to move longitudinally. In particular, the first DOF 200a includes the ultrasonic device 150 moving along a first axis A1. By way of example, FIG. 2A shows the ultrasonic device 150 located a first position P1 (denoted by solid lines) along the first axis A1 and at a second position P2 (denoted by dotted lines) along the first axis A1. However, the ultrasonic device 150 may be located at any position along the first axis A1. FIG. 2B illustrates a second DOF 200, 200b of the venipuncture device 100 that enables the ultrasonic device 150 to move vertically. In particular, the second DOF 200b includes the ultrasonic device 150 moving along a second axis A2. By way of example, FIG. 2B shows the ultrasonic device 150 located at a third position P3 (denoted by solid lines) along the second axis A2 and at a fourth position P4 (denoted by dotted lines) along the second axis A2. However, the ultrasonic device 150 may be located at any position along the second axis A2. FIG. 2C illustrates a third DOF 200, 200c of the venipuncture device 100 that enables the ultrasonic device 150 to rotate (e.g., enable yaw movement of the ultrasonic device 150). The third DOF 200c includes the ultrasonic device 150 rotating about a first focal point FP1. Here, the first focal point FP1 may indicate the direction of rotation of the ultrasonic device 150. By way of example, FIG. 2C shows the ultrasonic device 150 located at a fifth position P5 (denoted by solid lines) about the first focal point FP1 and at a sixth position P6 (denoted by dotted lines) about the first focal point FP1. However, the ultrasonic device 150 may be located at any position about the first focal point FP1.

FIG. 2D illustrates a fourth DOF 200, 200d of the venipuncture device 100 that enables the cannula positioning mechanism 134 to move laterally along a third axis A3. That is, the fourth DOF 200d is the cannula positioning mechanism 134 moving along the third axis A3. By way of example, FIG. 2D shows the cannula positioning mechanism 134 located at a first position P1 along the third axis A3 and a second position P2 along the third axis A3. However, the cannula positioning mechanism 134 may be located at any position along the third axis A3. FIG. 2E illustrates a fifth DOF 200, 200e of the venipuncture device 100 that enables the cannula holding mechanism 132 to move along a fourth axis A4. That is, the fifth DOF 200e is the cannula holding mechanism 132 moving along the fourth axis A4. By way of example, FIG. 2E shows the cannula holding mechanism 132 at a first position P1 along the fourth axis A4 and a second position P2 along the fourth axis A4. Notably, the first position P1 of the cannula holding mechanism 132 corresponds to a closed position that secures the cannula 130 within the cannula holding mechanism 132 while the second position P2 of the cannula holding mechanism 132 corresponds to an opened position that enables the cannula 130 to be inserted into, or removed from, the cannula holding mechanism 132. However, the cannula holding mechanism 132 may be located at any position along the fourth axis A4.

FIG. 2F illustrates a sixth DOF 200, 200f of the venipuncture device 100 that enables the cannula positioning mechanism 134 to move vertically along a fifth axis A5. That is, the sixth DOF 200f is the cannula positioning mechanism 134 moving along the fifth axis A5. By way of example, FIG. 2F shows the cannula positioning mechanism 134 located at a third position P3 along the fifth axis A5 and a fourth position P4 along the fifth axis A5. However, the cannula positioning mechanism 134 may be located at any position along the fifth axis A5. FIG. 2G illustrates a seventh DOF 200, 200g of the venipuncture device 100 that enables the cannula positioning mechanism 134 to rotate about a second focal point FP2. That is, the seventh DOF 200g is the cannula positioning mechanism 134 rotating about the second focal point FP2. By way of example, FIG. 2G shows the cannula positioning mechanism 134 located at a fifth position P5 about the second focal point FP2 and a sixth position P6 about the second focal point FP2. However, the cannula positioning mechanism 134 may be located at any position about the second focal point FP2. Here, the second focal point FP2 may indicate the direction of rotation of the ultrasonic device 150. FIG. 2H illustrates an eighth DOF 200, 200h of the venipuncture device 100 that enables the cannula positioning mechanism 134 to move along a sixth axis A6. That is, the eighth DOF 200h is the cannula positioning mechanism 134 moving along the sixth axis A6. By way of example, FIG. 2H shows the cannula positioning mechanism 134 located at a seventh position P7 along the sixth axis A6 and eighth position P8 along the sixth axis A6. However, the cannula positioning mechanism 134 may be located at any position along the sixth axis A6.

Before initiating the site selection process 300 described below, implementations herein may include a needle verification process, also referred to as needle tip sensing. The needle verification process occurs after the cannula 130 (referred to interchangeably as a needle) has been loaded into the cannula holding mechanism 132. The needle verification process is configured to verify the suitability of the loaded cannula 130 and determine the three-dimensional (3D) position of the tip of the cannula 130 relative to known datums on the venipuncture device 100, such as the cannula holding mechanism 142 and/or the ultrasonic device 150. The precise localization is advantageous because standard, off-the-shelf needles suitable for human use may have manufacturing tolerances that are insufficient for the high accuracy required by the venipuncture device 100, particularly concerning the distance and alignment between the cannula holding mechanism 132 and the actual tip of the cannula 130. That is, the venipuncture device 100 may require submillimeter accuracy for the tip of cannula 130 position relative to the ultrasound transducer within the ultrasonic device 150 to ensure accurate targeting during subsequent insertion.

To perform the needle verification process, the venipuncture device 100 may include a verification station or sensor arrangement, for example, housed within or integrated with the ultrasonic device 150 housing or another suitable location accessible by the cannula positioning mechanism 134. For instance, the verification station or sensor arrangement for performing the needle verification process may be housed in the needle sensing housing 131 of FIG. 1A. In one implementation, as shown in FIG. 1C, the sensor arrangement includes at least two optical sensors 136, 138, such as optical beam break sensors. The optical sensors 136, 138 may be mounted orthogonally (e.g., at approximately 90 degrees relative to each other) within the cannula holding mechanism 132, creating an intersecting sensing zone, akin to an “X” formed by the light beams. That is, the first optical sensor 136 may produce a first light beam 140 and the second optical sensor 138 produces a second light beam 142 forming the intersecting sensing zone. Upon loading the cannula 130, or upon receiving a command (e.g., from the data processing hardware 140 via the motor controller 172), the cannula positioning mechanism 134 moves the loaded cannula 130 towards and through this sensing zone. The needle verification process may involve multiple steps controlled by the data processing hardware 140. First, the cannula 130 is passed through the intersecting sensing zone (e.g., the “X: created by the orthogonal optical beams). As the cannula 130 interrupts each beam, the venipuncture device 100 registers the position of the cannula positioning mechanism 134. These two registered points, corresponding to the interruption of the two separate beams 140, 142 and relative to a known datum (e.g., the cannula holding mechanism 132), are used by the data processing hardware 140 to calculate a line representing the centerline axis of the loaded cannula 130.

Once the cannula 130 centerline is defined, the data processing hardware 140 instructs the cannula positioning mechanism 134 to drive the cannula 130 along this calculated centerline axis directly towards or into the sensors (or a designated sensing point). The point at which the very tip of the cannula 150 (e.g., the center of the cannula 130 tip lumen) interacts with or is detected by the sensor(s) is recorded. This provides a precise point along the previously determined centerline. Using the defined centerline and this endpoint, the data processing hardware 140 calculates the accurate three-dimensional (3D) coordinates of the cannula 130 tip relative to the cannula holding mechanism 132 and, by extension (given the known geometry), relative to the ultrasonic device 150 assembly. This calculated 3D cannula 130 tip position is stored in memory hardware 146 and is subsequently used as the reference position for the cannula 130 tip during the cannula 130 insertion phase.

Following the calculation of the 3D cannula 130 tip position, the data processing hardware 140 compares the calculated position against predetermined system tolerances or requirements stored in memory 146. These tolerances define an acceptable range for the location of the cannula 130 tip and orientation relative to the device components. If the calculated 3D position falls outside this allowable tolerance range, the cannula 130 load is rejected. Reasons for rejection may include the cannula 130 not being present, the cannula being outside allowable manufacturing tolerances (e.g., bent or incorrect length), or the cannula 130 being loaded improperly into the cannula holding mechanism 132. A rejection may trigger a notification to the operator via the user interface 170. Conversely, if the calculated 3D cannula tip position is within the acceptable system tolerance, the cannula load is accepted, and the precisely determined cannula tip coordinates are confirmed and stored for subsequent use. The venipuncture device 100 is then cleared to proceed with the next operational phase, typically the site selection process 300.

Referring now to FIG. 3, the site selection process 300 receives a sequence of image frames 154a, 154aa-an captured by the image capture device 150 (FIG. 1) moving across an anatomy portion of a subject. For example, the sequence of image frames 154 may correspond to ultrasound image frames 154 captured by the ultrasonic device 150. In some examples, the anatomy portion of the subject includes an arm of the subject. The ultrasonic device 150 may move across the anatomy portion of the subject over a predetermined distance. Each first ultrasound image frame 154a may capture one or more vessels 156 from the subject and/or one or more insufficient acoustic interface portions 158. In some examples, the first ultrasound image frame 154a includes only a portion of the one or more vessels 156. The vessels 156 are located beneath an exterior surface (i.e., skin) of the anatomy portion of the subject. As will become apparent, each of the one or more vessels 156 may represent a vein or an artery of the subject. The site selection process 300 is configured to process the sequence of first ultrasound image frames 154a to identify a candidate vessel 156, 156C to target for venipuncture from among the one or more vessels 156 captured by the sequence of first ultrasound image frames 154a. Simply put, the ultrasonic device 150 captures images of multiple vessels 156 of the subject and the site selection process 300 selects an optimal vessel 156 from the multiple captured vessels 156 to target for venipuncture.

In particular, the site selection process 300 includes a vessel identification (ID) model 310 that includes a deep neural network architecture. The vessel ID model 310 is configured to output vessel masks 312 based on a sequence of ultrasound image frames 154. Each vessel mask 312 corresponds to a respective one of the ultrasound image frames 154 and includes a representation of vessels 156 (if any) included in the respective one of the ultrasound image frames 154. Put another way, each vessel mask 312 denotes a location, size, and shape of any vessels 156 included in the corresponding ultrasound image frame 154 suitable for input to the site selection process 300. In particular, the vessel ID model 310 receives, as input, the sequence of first ultrasound image frames 154a and generates, as output, a respective first vessel mask 312, 312a for each of the first ultrasound image frames 154a. Notably, while the vessel ID model 310 receives the sequence of first ultrasound image frames 154a, the vessel ID model 310 may only receive a single first ultrasound image frame 154 from the sequence of first ultrasound image frames 154a at a time. The first vessel mask 312a indicates to the site selection process 300 where vessels 156 are located within each first ultrasound image frame 154a captured by the ultrasonic device 150. For each corresponding first ultrasound image frame 154a in the sequence of first ultrasound image frames 154a, the vessel ID model 310 processes the corresponding first ultrasound image frame 154a to generate the respective first vessel mask 312a that identifies one or more vessel portions 314 of the corresponding first ultrasound image frame 154a. That is, each of the one or more vessel portions 314 is a representation of where a respective vessel 156 (or portion of the respective vessel 156) is located within the corresponding first ultrasound image frame 154a. As such, for each respective first ultrasound image frame 154a that captured a respective vessel 156, the first vessel mask 312a generated by the vessel ID model 310 includes a respective vessel portion 314 indicating the presence and location of the respective vessel 156 within the respective first ultrasound image frame 154a. On the other hand, for each respective first ultrasound image frame 154a that does not capture any vessels 156, the first vessel mask 312a generated by the vessel ID model 310 does not include any vessel portions 314 because no vessels 156 are present within the respective first ultrasound image frame 154a.

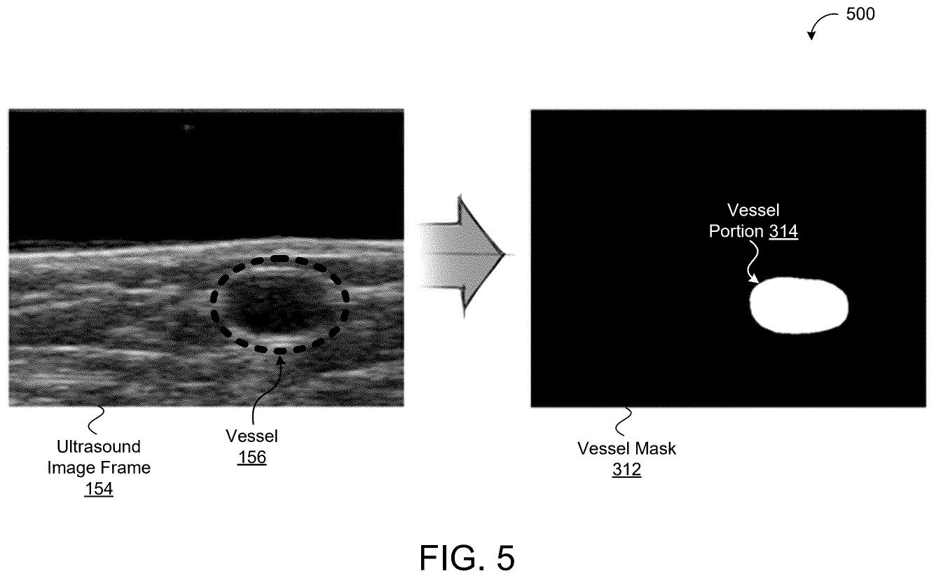

For example, FIG. 5 depicts a first graphical view 500 of an example ultrasound image frame 154 input into the vessel ID model 310 and a corresponding vessel mask 312 generated by the vessel ID model 310 based on the example ultrasound image frame 154. In the example shown, the example ultrasound image frame 154 includes a respective vessel 156, and thus, the corresponding vessel mask 312 output by the vessel ID model 310 includes a corresponding vessel portion 314 (e.g., denoted by the white circle) indicating the presence and location of the respective vessel 156 within the example ultrasound image frame 154. The location of the respective vessel 156 within the example ultrasound image frame 154 indicated by the vessel portion 314 may be a two-dimensional (2D) location, such as X-Y coordinates of corresponding pixels in the ultrasound image frame 154 that represent the vessel portion 314. Notably, the dashed circle around the vessel 156 of the example ultrasound image frame 154 is for the sake of clarity only, as it is understood that the vessel ID model 310 processes the example first ultrasound image frame 154 without any such annotation to identify the vessel 156 within the example ultrasound image frame 154.

Referring back to FIG. 3, in some scenarios, as the ultrasonic device 150 (FIG. 1) captures the sequence of first ultrasound image frames 154a, an insufficient acoustic interface portion 158 may exist between the acoustic interface (i.e., ultrasound sensor) 152 of the ultrasonic device 150 and the anatomy portion of the subject as the ultrasonic device 150 moves across the anatomy portion. A variety of different conditions may cause the insufficient acoustic interface portion 158. For example, the insufficient acoustic interface may be caused by the ultrasonic device 150 (FIG. 1) applying insufficient pressure to the anatomy portion of the subject and/or the ultrasonic device 150 moving across an uneven surface of the anatomy portion of the subject. As a result, the first ultrasound image frames 154a captured during the insufficient acoustic interface condition may be unreliable because of degraded image quality for the site selection process 300 to accurately identify whether vessels 156 are present (or not present) in each of the sequence of first ultrasound image frames 154a. For instance, a respective first ultrasound image frame 154a may tend to falsely indicate a presence of the vessel 156 within the respective first ultrasound image frame 154a because of the insufficient acoustic interface when, in fact, no vessels 156 are actually present. In other instances, a respective first ultrasound image frame 154a may tend to falsely indicate an absence of vessels 156 within the respective first ultrasound image frame 154a because of the insufficient acoustic interface when, in fact, one or more vessels 156 are actually present.

To that end, the site selection process 300 employs a contact detection model 320 that is configured to generate contact masks 322 based on the sequence of ultrasound image frames 154. That is, the contact detection model 320 receives, as input, the sequence of first ultrasound image frames 154a and generates, as output, first contact masks 322, 322a. In particular, for each corresponding first ultrasound image frame 154a in the sequence of first ultrasound image frames 154a, the contact detection model 320 processes the corresponding first ultrasound image frame 154a to generate a respective first contact mask 322a that identifies one or more insufficient contact portions 324 of the corresponding first ultrasound image frame 154a. That is, the one or more insufficient contact portions 324 each indicate a presence and location of an insufficient acoustic interface (if any) within the corresponding first ultrasound image frame 154a. The insufficient contact portion 324 may correspond to an entirety of the first ultrasound image frame 154a or only a portion of the first ultrasound image frame 154a. In short, each insufficient contact portion 324 indicates a corresponding portion of a respective first ultrasound image frame 154a that the site selection process 300 is unable to accurately rely upon when identifying the candidate vessel 154C to target for venipuncture. In some examples, the contact detection model 320 outputs contact masks 322 only when the contact detection model 320 identifies the presence of insufficient contact portions 324, but otherwise does not output contact masks 322. Thus, in these examples, the contact detection model 320 does not output any contact masks 322 for ultrasound image frames 154 that do not include insufficient acoustic interface portions 158. In other examples, the contact detection model 320 outputs contact masks 322 regardless of whether the contact detection model 320 identifies the presence of insufficient contact portions. For instance, the contact detection model 320 may output an entirely black contact masks 322 when there are no insufficient contact portions.

In some implementations, the vessel ID model 310 includes a first deep neural network architecture configured to receive, as input, the sequence of ultrasound image frames 154 and generate, as output, the vessel masks 312 and the contact detection model 320 includes a second deep neural network architecture different from the first neural network and configured is configured to receive, as input, the sequence of ultrasound image frames 154 and generate, as output, the contact masks 322. Simply put, the vessel ID model 310 includes the first deep neural network architecture and the contact detection model 320 includes the second deep neural network architecture different than the first deep neural network architecture. In other examples, the vessel ID model 310 and the contact detection model 320 each include a same deep neural network architecture configured to receive, as input, the sequence of ultrasound image frames 154 and generate, as output, both the vessel masks 312 and the contact masks 322. That is, a single deep neural network architecture includes both the vessel ID model 310 and the contact detection model 320.

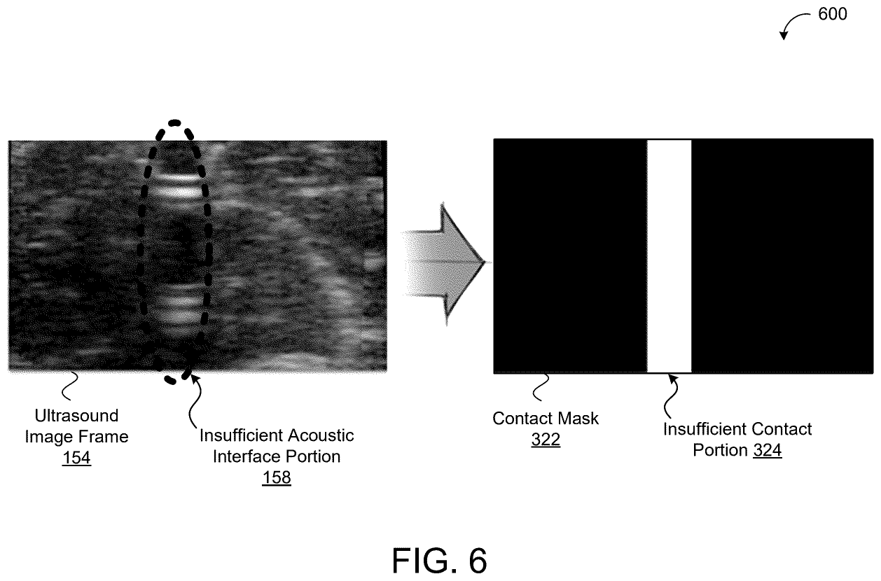

FIG. 6 depicts a second graphical view 600 of another example ultrasound image frame 154 input into the contact detection model 320 and a corresponding contact mask 322 generated by the contact detection model 320 based on the example ultrasound image frame 154. Notably, in the example shown, the example ultrasound image frame 154 includes an insufficient acoustic interface portion 158, and thus, the corresponding contact mask 322 output by the contact detection model 320 includes a corresponding insufficient contact portion 324 (e.g., denoted by the white portion of the corresponding contact mask 322) indicating the presence and location of the insufficient acoustic interface portion 158 within the example ultrasound image frame 154. The location of the insufficient contact portion 324 within the example ultrasound image frame 154 may be a 2D location, such as X-Y coordinates of corresponding pixels in the ultrasound image frame 154 that represent the insufficient contact portion 324. Here, the insufficient contact portion 324 corresponds to only a portion of the example ultrasound image frame 154. As will become apparent, the site selection process 300 would only process the portions of the example ultrasound image frame 154 that correspond to the black portions of the contact mask 322 (shown in FIG. 6) and discard the portions of the example ultrasound image frame 154 corresponding to the white portion (e.g., the insufficient contact portion 324 shown in FIG. 6). Notably, the dashed circle around the insufficient acoustic interface portion 158 of the example ultrasound image frame 154 is for the sake of clarity only, as it is understood that the contact detection model 320 processes the example ultrasound image frame 154 without any such annotation to identify the insufficient acoustic interface portion 158 within the example ultrasound image frame 154.

Referring back to FIG. 3, the site selection process 300 employs a validation module 330 that is configured to generate a validated vessel mask 312, 312V based on the vessel mask 312 received from the vessel ID model 310 and the contact mask 322 (if any) received from the contact detection model 320. That is, the validation module 330 receives, as input, the respective first vessel mask 312a generated by the vessel ID model 310 and respective first contact mask 322a generated by the contact detection model 320 and outputs a first validated vessel mask 312V, 312Va. Here, the respective first vessel mask 312a and the respective first contact mask 322a received by the validation module 330 are each generated based on a same corresponding first ultrasound image frame 154a in the sequence of first ultrasound image frames 154a. Thus, the validation module 330 compares the respective first vessel mask 312a and the respective first contact mask 322a to determine whether the respective first contact mask 322a includes any insufficient contact portions 324 that overlap with any of the vessel portions 314 identified by the respective first vessel mask 312a in the same corresponding first ultrasound image frame 154a. The validation module 330 validates the respective first vessel mask 312a by discarding any vessel portions 314 identified by the respective first vessel mask 312a that overlap with insufficient contact portions 324 identified by the respective first contact mask 322a. That is, discarded vessel portions 314 are not considered by the site selection process 300 to identify the candidate vessel 156C to target for venipuncture.

Advantageously, discarding vessel portions 314 that overlap with insufficient contact portions 324 prevents the site selection process 300 from inaccurately selecting the candidate vessel 156C based on a respective first ultrasound image frame 154a captured during an insufficient acoustic interface condition. On the other hand, when a respective first ultrasound image frame 154a does not include any insufficient acoustic interface portion 158, the contact detection model 320 does not generate the contact mask 322. Therefore, the validation module 330 does not discard any vessel portions 314 from the first vessel mask 312a such that the first validated vessel mask 312Va output by the validation module 330 is the same as the first vessel mask 312a output by the vessel ID model 310. Here, the first vessel mask 312a output by the vessel ID model 310 may bypass the validation module 330 because the first vessel mask 312a output by the vessel ID model 310 and the first validated vessel mask 312Va are the same. Thus, the first vessel mask 312a and the first validated vessel mask 312Va may be used interchangeably herein.

Each respective first ultrasound image frame 154a of the sequence of first ultrasound image frames 154a is paired with corresponding first three-dimensional position data 153a of the ultrasonic device 150 (FIG. 1) when the ultrasonic device 150 captured the corresponding first ultrasound image frame 154a. For instance, the corresponding first three-dimensional position data 153a of the ultrasonic device 150 may include a three-dimensional XYZ coordinate corresponding to a location of the ultrasonic device 150 when the ultrasonic device 150 captured a respective first ultrasound image frame 154a. In some examples, the first three-dimensional position data 153a includes a pose (e.g., that indicates translation and rotation) of the ultrasonic device 150 when the corresponding first ultrasound image frame 154a was captured. Advantageously, the pairing of the first three-dimensional position data 153a with each respective first ultrasound image frame 154a enables the site selection process 300 to determine a three-dimensional location (e.g., XYZ coordinate) of any vessel portions 314 identified by the vessel ID model 310 from the two-dimensional first ultrasound image frames 154a.

Accordingly, the site selection process 300 employs a vessel map generator 340 that is configured to generate a three-dimensional vessel structure map 700 based on the vessel masks 312 (or validate vessel masks 312V). In particular, the vessel map generator 340 receives, as input, the first vessel masks 312a (or first validated vessel masks 312Va) generated for the sequence of first ultrasound image frames 154a and the corresponding first three-dimensional position data 153a and generates, as output, a first three-dimensional vessel structure map 700, 700a that represents the vessels 156 within the anatomy portion of the subject. That is, the vessel map generator 340 processes the first vessel masks 312a including vessel portions 314 associated with a two-dimensional location within a respective first ultrasound image frame 154a (e.g., two-dimensional image) and the corresponding first three-dimensional position data 153a paired with the respective first ultrasound image frame 154a, to generate the first three-dimensional vessel structure map 342a that represents vessels 156 within the anatomy portion of the subject. Put another way, the vessel map generator 340 processes the first vessel masks 312a and the corresponding first three-dimensional position data 153a for each of the sequence of first ultrasound image frames 154a and generates the first three-dimensional vessel structure map 700a that includes a three-dimensional representation of all the first vessel masks 312a identified by the vessel ID model 310 and validated by the validation module 330 from the sequence of first ultrasound image frames 154a. For instance, the vessel map generator 340 may generate the first three-dimensional vessel structure map 700a by stitching together each first vessel mask 312a using the corresponding first three-dimensional position data 153a of each first ultrasound image frame 154a. Thus, the first three-dimensional vessel structure map 700a is a three-dimensional representation of vessels 156 from the anatomy portion of the subject that the site selection process 300 may target for venipuncture.

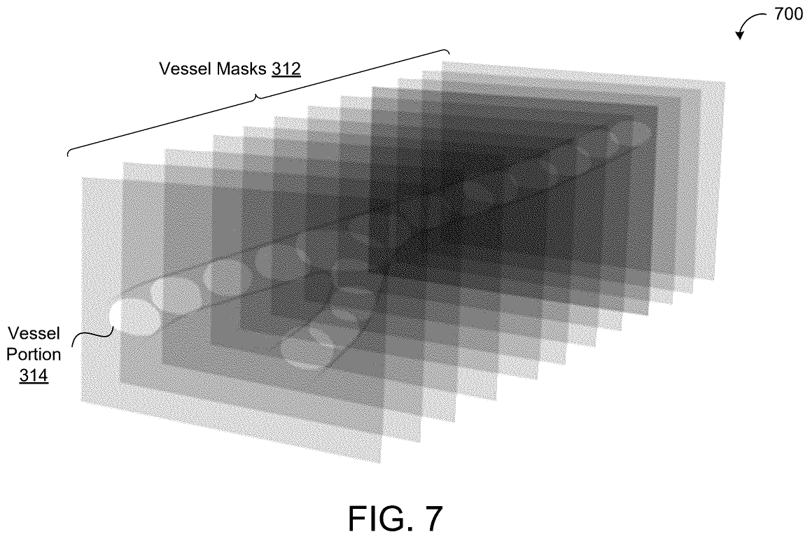

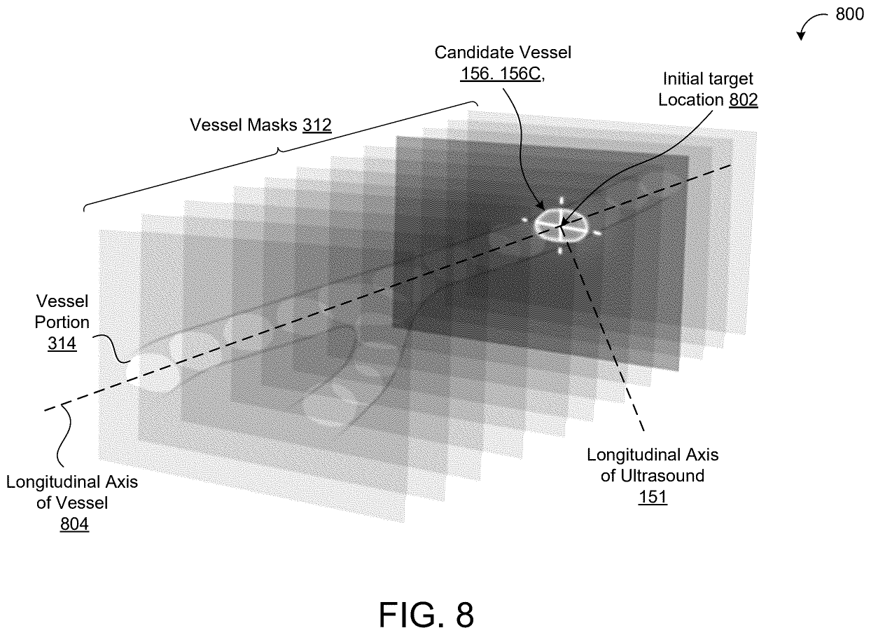

FIG. 7 depicts an example three-dimensional vessel structure map 700 output by the vessel map generator 340. As shown in FIG. 7, the example three-dimensional vessel structure map 700 includes twelve (12) vessel masks 312 stitched together with each respective vessel mask 312 including at least one respective vessel portion 314 representing a corresponding vessel 156 within the anatomy portion of the subject. By stitching together each two-dimensional vessel mask 312 using the corresponding three-dimensional position data 153, the three-dimensional vessel structure map 700 forms a three-dimensional representation of each vessel portion 314 identified by the vessel ID model 310 whereby each vessel portion 314 is associated with a respective three-dimensional location (e.g., three-dimensional XYZ coordinate). As such, the site selection process 300 can target the associated three-dimensional location of the vessel 156 selected as the candidate vessel 156C (FIG. 8).

Referring back to FIG. 3, the first three-dimensional vessel structure map 700a is a three-dimensional representation of possible vessels 156 that the site selection process 300 may target for venipuncture. To that end, the site selection process 300 selects an optimal vessel 156 from among the possible vessels 156 of the first three-dimensional vessel structure map 700a for venipuncture. In particular, site selection process 300 employs a site selector 350 that is configured to receive the first three-dimensional vessel structure map 700a generated by the vessel map generator 340 and output a three-dimensional site selection map 800 that includes the candidate vessel 156C and a corresponding initial target location 802 (e.g., XYZ coordinate) of the candidate vessel 156C to target for venipuncture. That is, the three-dimensional site selection map 800 is similar to the three-dimensional vessel structure map 700, but further includes the selected candidate vessel 156C from among the plurality of vessels 156 and the initial target location 802 associated with the selected candidate vessel 156C. For instance, the initial target location 802 may represent a center location of the selected candidate vessel 156C.

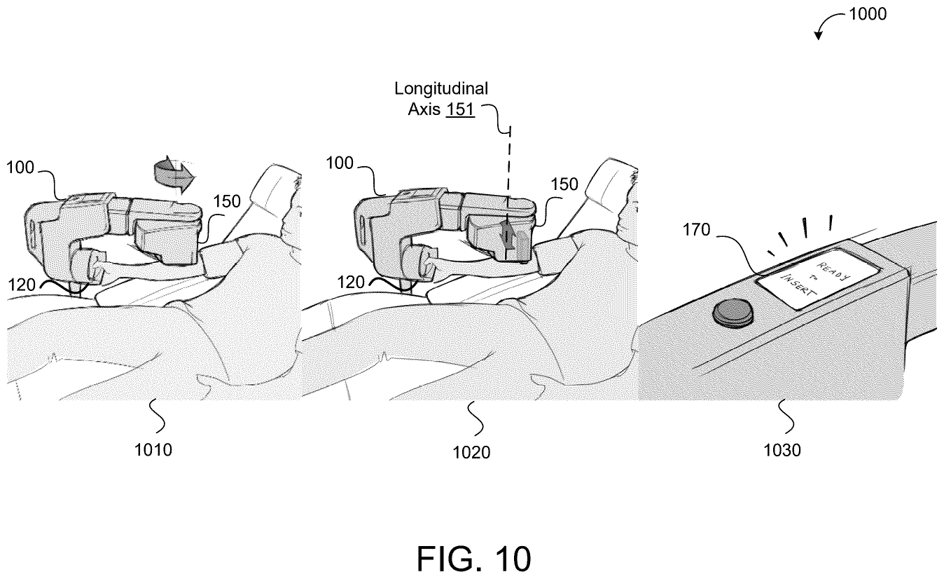

For example, FIG. 8 depicts an example three-dimensional site selection map 800 output by the site selector 350. As shown in FIG. 8, the example three-dimensional site selection map 800 includes twelve (12) vessel masks 312 each including at least one respective vessel portion 314. Moreover, the example three-dimensional site selection map 800 includes a selected candidate vessel 156C and the associated initial target location 802 of the selected candidate vessel 156C. Here, the candidate vessel 156C represents an optimal vessel from among the vessels 156 (e.g., represented by vessel portions 314 of the vessel masks 312) to target for venipuncture. The example three-dimensional site selection map 800 may include a longitudinal axis 804 of the candidate vessel 156C (as well as the longitudinal axis of other vessels). Moreover, as described in greater detail with reference to FIGS. 4 and 10, the longitudinal axis 151 of the ultrasonic device 150 may be substantially perpendicular to the longitudinal axis 804 of the candidate vessel 156C such that the ultrasonic device 150 applies a force upon the candidate vessel 156C that is substantially perpendicular.

Referring back to FIG. 3, in particular, the site selector 350 processes the three-dimensional vessel structure map 700 to select, from the vessels 156 represented by vessel portions 314 in the first three-dimensional vessel structure map 700a, the candidate vessel 156C. More specifically, the site selector 350 processes the first three-dimensional vessel structure map 700a to identify the plurality of vessels 156 within the anatomy portion of the subject and extracts respective vessel properties 352 from each corresponding vessel 156 of the plurality of vessels 156 identified. Here, the respective vessel properties 352 extracted from each corresponding vessel 156 includes at least one of a diameter of the corresponding vessel 156, an angle of the corresponding vessel 156 relative to a reference angle (e.g., angle between the corresponding vessel 156 and a current pose of the ultrasonic device 150 and/or cannula 130 (FIG. 1A)), a depth of the corresponding vessel 156 from an exterior surface of the anatomy portion, or locations (in the three-dimensional space) any branch vessels branching from the corresponding vessel 156.

For each corresponding vessel 156 from the three-dimensional vessel structure map 700, the site selector 350 determines a respective score 354 for the corresponding vessel 156 based on the extracted vessel properties 352 of the corresponding vessel 156 and a set of predefined criteria 355. For example, the predefined criteria 355 may indicate rules for the site selector 350 to assign higher scores to vessels 156 with vessel properties 352 representing a larger diameter, a larger distance between other surrounding vessels 156, a shallower depth from the exterior surface of the anatomy, and/or straight vessels (as opposed to curved vessels). Thereafter, the site selector 350 ranks each corresponding vessel 156 from the three-dimensional vessel structure map 700 based on the determined scores 354 and selects the corresponding vessel 156 having the highest rank (e.g., highest score 354) as the candidate vessel 156C to target for venipuncture. That is, the selected candidate vessel 156C has the optimal qualities for venipuncture determined based on the vessel properties 352 and the predefined criteria. In some examples, the predefined criteria 355 is configurable to bias the site selector 350 to select candidate vessels 156C with a certain set of vessel properties 352 (e.g., a set of properties that correspond/enable successful venipuncture of the subject).

The site selection process 300 may instruct the image capture device (e.g., ultrasonic device) 150 to move to a target position against the anatomy portion of the subject based on the initial target location 802 of the candidate vessel 156C of the three-dimensional site selection map 800. Here, instructing the image capture device 150 to move to the target position includes instructing the image capture device 150 to move to a target orientation that aligns the longitudinal axis 151 of the image capture device 150 in a direction substantially perpendicular to the longitudinal axis 804 (FIG. 8) of the candidate vessel 156C at the target location. For instance, the data processing hardware 140 may cause the motor controller 172 to instruct the one or more motors 174 to move the venipuncture device 100 to the target position (FIG. 1B). The target position corresponds to a position of the ultrasonic device 150 when the ultrasonic device 150 captured the respective first ultrasound image frame 154a that includes the vessel 156 the site selection process 300 selected as the candidate vessel 156C. Alternatively, the target position corresponds to a position of the image capture device 150 when the image capture device 150 captured the respective first image frame 154a that includes the vessel 156 the site selection process 300 selected as the candidate vessel 156C. As such, the target position may be derived from the corresponding first three-dimensional position data 153a when the ultrasonic device 150 captured the respective first ultrasound image frame 154a that includes the candidate vessel 156C.

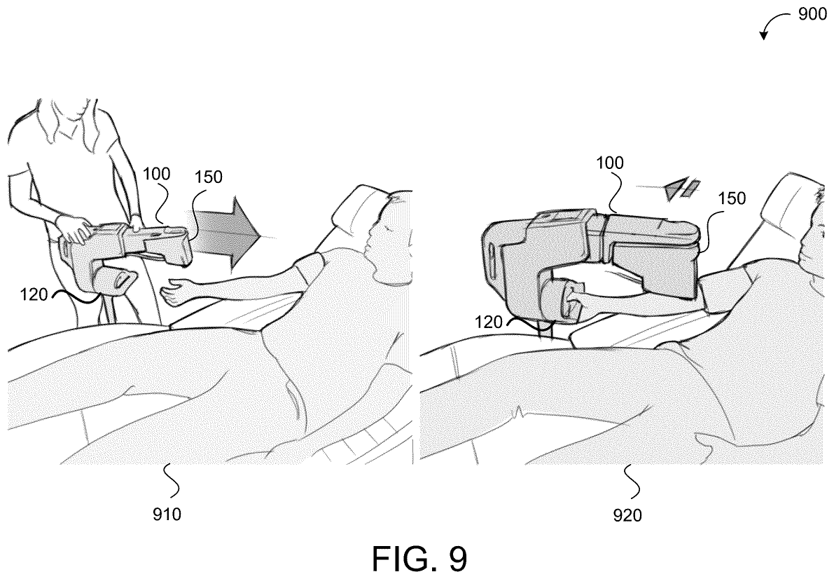

FIG. 9 illustrates images 910, 920 that depict the venipuncture device 100 performing the site selection process 300 (FIG. 3). In particular, a first image 910 depicts an operator of the venipuncture device 100 moving the venipuncture device 100 towards the anatomy portion of the subject to capture ultrasound image frames 154. Moreover, a second image 920 shows the subject grasping the grip handle 120 as the ultrasonic device 150 moves along the anatomy portion (i.e., arm) of the subject while capturing ultrasound image frames 154 for processing by the site selection process 300.

Some venipuncture devices 100 may be constructed with additional optimization features that operate as a means to certify the candidate vessel 156C identified by the site selection process 300. For example, it may be advantageous to certify the candidate vessel 156C because the patient may have moved their arm from the time the ultrasonic device 150 captures the ultrasound image frame 154 to the time when the venipuncture device 100 determines the candidate vessel 156C. In some implementations, the venipuncture device 100 includes a set of sensors that tracks the location of the patient's arm (e.g., starting from when the venipuncture device 100 initially captures the sequence of first ultrasound image frames 154a) such that any movement by the patient can be taken into account and therefore reconciled with the location of the candidate vessel 156C (e.g., modify the location by positional data or a movement vector detected by the set of sensors). Additionally or alternatively, informed by the candidate vessel 156C, the venipuncture device 100 may repeat some version of operations performed during the site selection process 300 as a confirmation process to generate a final location to perform the venipuncture on the patient. In some examples, an optimization feature may be to confirm that the candidate vessel 156C corresponds to a vein rather than an artery because although the site selector 350 may be biased to select a candidate vessel 156C that corresponds to a vein (e.g., by the properties 352 and/or criteria 355), that bias could have a margin of error, which could be abated by further confirmation.

Referring back to FIG. 3, after identifying the candidate vessel 156C, the venipuncture device 100 confirms the candidate vessel 156C is suitable for puncturing using the vein confirmation process 400 (FIG. 4). The vein confirmation process 400, however, is optional. That is, the venipuncture device 100 may execute the vein confirmation process 400 independent from, or in combination with, the site selection process 300. For example, after identifying the candidate vessel 156C, the venipuncture device 100 may instruct the venipuncture device 100 to insert the cannula into the candidate vessel 156C without executing the vein confirmation process 400. However, in some scenarios, after selecting the candidate vessel 156C, but before puncturing, the patient may have moved their arm (e.g., even moving a few millimeters) such that the initial target location 802 no longer aligns with the candidate vessel 156C. To be clear, a human viewing the patient's arm would likely not be able to decipher that the patient has moved his or her arm out of alignment with the initial target location 802 since the amount of movement may be ever so slight. Additionally, the candidate vessel 156C selected by the site selection process 300 could be an artery rather than a vein. Notably, venipuncture requires puncturing veins and not arteries. If an artery is punctured rather than a vein during venipuncture the patient may be harmed. Processing of the three-dimensional site selection map 800 may not confidently decipher vessels 156 that are veins from those that are arteries. Thus, in these scenarios, the venipuncture device 100 may confirm whether the candidate vessel 156C is truly an artery rather than a vein and/or whether the candidate vessel 156C is still in the initial target location 802 (e.g., the patient has not moved since identifying the initial target location 802) before puncturing the candidate vessel 156C.

Referring now to FIG. 4, the vein confirmation process 400 is configured to confirm the candidate vessel 156C selected during the site selection process 300 (FIG. 3) for use by the venipuncture device 100 for venipuncture. Once the ultrasonic device 150 is at the target position, the vein confirmation process 400 instructs the ultrasonic device 150 to apply pressure against the anatomy portion of the subject to exert a force upon the candidate vessel 156C at the target location. Here, the ultrasonic device 150 applies pressure from the target position and against the anatomy portion of the subject. More specifically, the vein confirmation process 400 instructs the ultrasonic device 150 to increase pressure from an initial pressure value to a final pressure value during a predetermined duration of time. Moreover, the ultrasonic device 150 captures a sequence of second ultrasound image frames 154, 154ba-bn while the ultrasonic device 150 is applying the pressure against the anatomy portion of the subject from the target position. That is, the second sequence of ultrasound image frames 154b represent image frames captured while the ultrasonic device 150 applies pressure from the target position based on the initial target location 802 of the candidate vessel 156C. During the site selection process 300 (FIG. 3) and the vein confirmation process 400 the ultrasonic device 150 captures a sequence of ultrasound image frames 154 that enable the functionality of the respective process. Generally, the first sequence of ultrasound image frames 154a refer to ultrasound image frames 154 captured by the ultrasonic device 150 during the site selection process 300 while the second sequence of ultrasound image frames 154b refer to ultrasound image frames 154 captured by the ultrasonic device 150 during the vein confirmation process 400. Although each process 300, 400 has different operations, the properties of each ultrasound image frame 154 captured by the ultrasonic device 150 or generated by the venipuncture device 100 may be similar or relatively identical even though the ultrasound image frames 154 are being used by different processes. Yet, it is also contemplated that the venipuncture device 100 may modify or optimize the ultrasound image frames 154 depending on the particular process 300, 400 that the ultrasound image frames 154 were captured during. Similarly, each process 300, 400 may leverage vessel masks 312, contact masks 322, and/or validated vessel masks 312V. For the sake of clarity, a vessel mask 312, a contact mask 322, and/or a validated vessel mask 312V may be designated as a “first” generally indicating that it stems from the site selection process 300 whereas, if designated as a “second,” generally indicating that it stems from the vein confirmation process 400. That is, the quantitative modifier of “first” or “second” is used to aid an understanding of which process the element is associated with.

In some implementations, the vein confirmation process 400 instructs an auxiliary component, separate from the ultrasonic device 150, to apply pressure against the anatomy portion of the subject to exert a force upon the candidate vessel 156C at the target location. That is, the auxiliary component may be another component of the venipuncture device 100 that is in communication with the ultrasonic device 150 that applies pressure against the anatomy portion of the subject while the ultrasonic device 150 captures the second sequence of ultrasound image frames 154b. In these implementations, the auxiliary component may apply the pressure at or distill from the target position while the ultrasonic device 150 captures the second sequence of ultrasound image frames 154b at the target position.

The vein confirmation process 400 processes the sequence of second ultrasound image frames 154b captured while the ultrasonic device 150 is applying pressure against the anatomy portion at the target location 802 of the candidate vessel 156C to ensure that the candidate vessel 156C is a vein and that the initial target location 802 from the site selection process 300 still corresponds to a center point of the candidate vessel 156C. Notably, the respective second vessel mask 312 generated for at least the initial second ultrasound image frame 154b captured before applying the downward pressure may be compared to the respective first vessel mask 312 from which the initial target location 802 was obtained to determine whether the initial target location 802 is no longer aligned with the candidate vessel 156C, thereby requiring the venipuncture device 100 to adjust its pose accordingly.

In particular, the vein confirmation process 400 employs the vessel ID model 310 that receives, as input, the sequence of second ultrasound image frames 154b and generates, as output, a respective second vessel mask 312, 312b for each of the second ultrasound image frames 154b. For each corresponding second ultrasound image frame 154b in the sequence of second ultrasound image frames 154b, the vessel ID model 310 processes the corresponding second ultrasound image frame 154b to generate the respective second vessel mask 312b that identifies one or more vessel portions 314 of the corresponding second ultrasound image frame 154b. That is, a vessel portion 314 includes a representation of where the candidate vessel 156C is located within the corresponding second ultrasound image frame 154b. As such, for each respective second ultrasound image frame 154b that captured the candidate vessel 156C, the second vessel mask 312b generated by the vessel ID model 310 includes a respective vessel portion 314 indicating the presence and location of the candidate vessel 156C within the respective second ultrasound image frame 154b.

In some scenarios, as the ultrasonic device 150 (FIG. 1) captures the sequence of second ultrasound image frames 154b, the insufficient acoustic interface portion 158 exists between the acoustic interface (i.e., ultrasound sensor) 152 of the ultrasonic device 150 and the anatomy portion of the subject. To that end, the vein confirmation process 400 employs the contact detection model 320 that receives, as input, the sequence of second ultrasound image frames 154b and generates, as output, second contact masks 322, 322b. The vein confirmation process 400 may optionally employ the contact detection model 320 to generate the second contact masks 322, 322b since there is already a high level of confidence of where the candidate vessel 156C is located since the sequence of second ultrasound image frames 154b are all captured from the initial target location 802. The vein confirmation process 400 may optionally employ the validation module 330 to validate each respective second vessel mask 312b by discarding any vessel portions 314 identified by the respective second vessel mask 312b that overlap with insufficient contact portions 324 identified by the respective second contact mask 322b. In scenarios when second contact masks 322b are not generated, all of the second vessel masks 312b are retained and are assumed valid.

Each respective second ultrasound image frame 154b of the sequence of second ultrasound image frames 154b is paired with corresponding second three-dimensional position data 153, 153b of the ultrasonic device 150 (FIG. 1) when the ultrasonic device 150 captured the corresponding second ultrasound image frame 154b. For instance, the corresponding second three-dimensional position data 153b of the ultrasonic device 150 may include a three-dimensional XYZ coordinate corresponding to a location of the ultrasonic device 150 when the ultrasonic device 150 captured a respective second ultrasound image frame 154b. In some examples, the second three-dimensional position data 153b includes a pose of the ultrasonic device 150 when the corresponding second ultrasound image frame 154b was captured.

The vein confirmation process 400 includes a vein confirmation model 410 configured to receive, as input, the sequence of validated second vessel masks 312Vb to extract compressive properties 412 of the candidate vessel 156C. Put another way, the vein confirmation model 410 processes the sequence of second ultrasound image frames 154b as the ultrasonic device 150 applies pressure to the anatomy portion of the subject and extracts the compressive properties 412 of the candidate vessel 156C from the sequence of validated vessel masks 312Vb. Thus, the vein confirmation model 410 receives probe forces 162 (e.g., from the pressure sensor 160 (FIG. 1B)) representing a magnitude of force exerted upon the candidate vessel 156C at the target location. As such, each second ultrasound image frame 154b is paired with a corresponding probe force 162. In some examples, the vein confirmation model 410 is configured to extract pulsation properties of the candidate vessel 156C in addition to, or in lieu of, the compressive properties 412. Thus, in these examples, the vein confirmation model 410 may distinguish veins from arteries based on pulsation properties of the candidate vessel 156C since veins and arteries have different pulsation properties.

The vein confirmation model 410 generates a classification output 415 indicating whether or not the candidate vessel 156C is a vein or an artery based on the compressive properties 412 and the probe forces 162 exerted upon the candidate vessel 156C. That is, when a sufficient force is exerted upon a vein, the vein will compress while a same force would not cause an artery to compress. Accordingly, the vein conformation model 410 can classify the candidate vessel 156C as an artery or a vein by monitoring the compressive properties of the candidate vessel 156C as the venipuncture device 100 applies a force to the candidate vessel 156C. The classification output 415 indicates whether the vein confirmation model 410 classifies the candidate vessel 156C as a vein or an artery.

For instance, the vein confirmation model 410 is trained to classify vessels 156 as a vein when the compressive properties 412 of the candidate vessel 156C indicate a decreasing cross-sectional area of the corresponding vessel portion 314 in the validated second vessel masks 312Vb responsive to increases in magnitude of force exerted upon the candidate vessel 156C. That is, when the cross-sectional area (i.e., diameter) of the vessel 156 (as represented by the corresponding vessel portion 314 in the second vessel masks 312Vb) decreases such that the cross-sectional area satisfies a threshold value, the vein confirmation model 410 classifies the vessel 156 as a vein. On the other hand, the vein confirmation model 410 is trained to classify a vessel 156 as an artery when the compressive properties 412 of the vessel 156 indicates that the cross-sectional area does not decrease responsive to increases in the magnitude of force. Stated differently, when the cross-sectional area of the vessel 156 fails to satisfy the threshold value, the vein confirmation model 410 classifies the vessel as an artery.

In some implementations, in response to the vein confirmation model 410 classifying the candidate vessel 156C as an artery (e.g., not suitable for venipuncture), the site selection process 300 is repeated to select a new candidate vessel 156C. Here, the vein confirmation process 400 then determines whether the new candidate vessel 156C is a vein or an artery. In other implementations, in response to the vein confirmation model 410 classifying the candidate vessel 156C as an artery, the vein confirmation process 400 instructs the image capture device 150 to move to a target position against the anatomy portion of the subject based on another target location 802 associated with another candidate vessel 156C. Thereafter, the vein confirmation process 400 is repeated at the other target location 802 associated with the other candidate vessel 156C. Here, the other candidate vessel 156C may be the second highest ranked vessel 156 identified by the site selection process 300 (FIG. 3). Advantageously, by moving to the second highest ranked vessel 156, the vein confirmation process 400 avoids repeating the entire site selection process 300 while still selecting another vessel 156 to target for venipuncture that has a high determined score 354 (FIG. 3).

On the other hand, in response to the vein confirmation model 410 classifying the candidate vessel 156C as a vein (e.g., suitable for venipuncture), the vein confirmation model 410 sends the classification output 415 to a position selector 420 configured to instruct the cannula positioning mechanism 134 (e.g., cannula positioning device) (FIG. 1A) to insert the cannula 130 into the candidate vessel 156 that includes the vein confirmed by the vein confirmation model 410. More specifically, based on determining the candidate vessel 156C is a vein, the position selector 420 instructs the ultrasonic device 150 to capture, from the target position against the anatomy portion of the subject, an addition ultrasound image frame (e.g., third ultrasound image frame) 154, 154c. Moreover, the position selector 420 may process the third ultrasound image frame 154c to identify the candidate vessel 156 and determine a final target location 422 (e.g., XYZ coordinate) of the candidate vessel 156C to puncture. Here, the final target location 422 may be a center of the candidate vessel 156C. Thus, the position selector 420 outputs instructions 424 including the final target location 422 that instructs the cannula positioning device 134 to insert the cannula 130 into the candidate vessel 156C at the final target location 422. For instance, the position selector 420 may output the instructions 424 to the data processing hardware 140 and/or the motor controller 172.

In some implementations, the vein confirmation process 400 generates a corresponding vessel mask 312 and a corresponding contact mask 322 based on the third ultrasound image frame 154c and generates a validated vessel mask 312V based on the corresponding vessel mask 312 the corresponding contact mask 322. Thus, in these implementations, the vein confirmation process 400 selects the final target location based on position data 153 associated with the ultrasonic device 150 when the ultrasonic device 150 captured the third ultrasound image frame 154c.

In some implementations, the vein confirmation model 410 monitors other inputs in addition to, or in lieu of, the second sequence of ultrasonic image frames 154b. For instance, during the site selection process 300 the venipuncture device 100 may obtain pressure data (e.g., from the pressure sensor 160) associated with the candidate vessel 156C. The pressure data may represent pressures between the subject and the pressure sensor 160 at the target position. Additionally or alternatively, the venipuncture device may obtain position data associated with the candidate vessel 156C. The position data may represent a position of the subject's arm during each process 300, 400. As such, during the vein confirmation process 400, the vein confirmation model 410 may compare pressure data and/or the position data with the ultrasonic device 150 at the target position. Here, any discrepancies between the pressure data and/or position data obtained during the processes 300, 400 may indicate that the subject has moved their arm after the candidate vessel 156C was identified. As such, the vein confirmation process 400 may initiate the site selection process 300 to re-execute.

FIG. 10 depicts a sequence of images 1010, 1020, 1030 that depict the venipuncture device 100 performing the vein confirmation process 400 (FIG. 4). For instance, a first image 1010 shows the venipuncture device 100 moving the ultrasonic device 150 to the target location against the anatomy portion (i.e., arm) of the subject where the ultrasonic device 150 was located when it captured the ultrasound image frame 154 including the candidate vessel 156C. Thereafter, a second image 1020 depicts the ultrasonic device 150 applying a pressure from the target position and target orientation against the candidate vessel 156C to confirm the candidate vessel 156 is a vein. Here, the target orientation may align the longitudinal axis 151 of the ultrasound image device 150 in a direction substantially perpendicular to the longitudinal axis 804 (FIG. 8) of the candidate vessel 156C at the target location. Thus, instructing the ultrasonic device 150 to apply pressure includes applying pressure in the direction that is substantially perpendicular to the longitudinal axis 804 of the candidate vessel 156C.

A third image 1030 depicts the user interface 170 displaying to the operator a notification indicating that the candidate vessel 156C is suitable for venipuncture (e.g., confirmation that the candidate vessel 156C is a vein). Thus, the operator may provide a user input that instructs the venipuncture device 100 to puncture the candidate vessel 156C. Alternatively, the venipuncture device 100 may puncture the candidate vessel 156C based on confirming the candidate vessel 156C is a vein without any operator input. The venipuncture device 100 instructs the cannula positioning device 134 (FIG. 2) to insert the cannula 130 into the candidate vessel 156C while the cannula axis (e.g., longitudinal axis of the cannula 130) 131 (FIG. 2) is oriented at a target angle relative to the longitudinal axis 804 (FIG. 8) of the vein. The venipuncture device 100 may instruct the cannula 130 to operate (e.g., operate using the degrees of freedom depicted in FIG. 2) to target the final target location 422 at the target angle. Here, the target angle may be such that the cannula axis 131 is substantially perpendicular to the longitudinal axis 804 of the vein. However, the target angle may be any suitable angle.

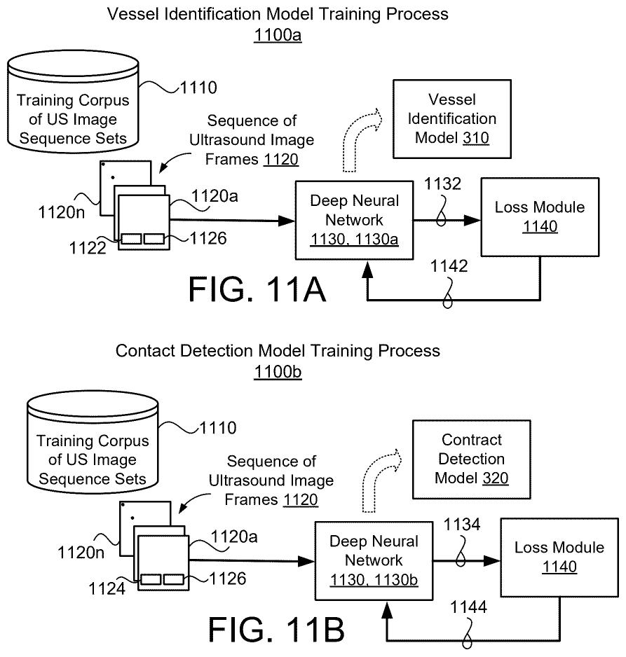

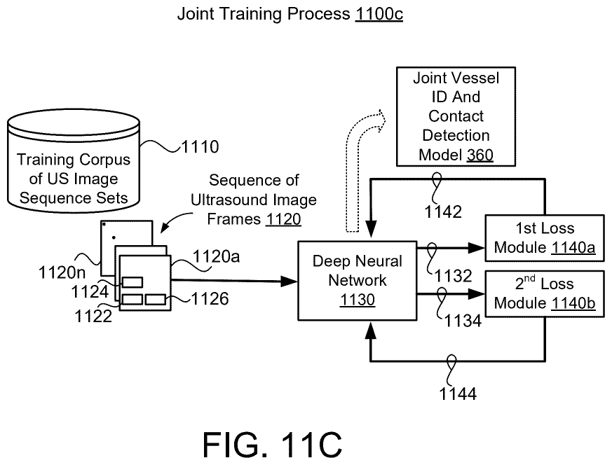

FIG. 11A shows an example vessel identification (ID) model training process 1100, 1100a that may be used to train the vessel ID model 310. The training process 1100a may execute on data processing hardware of a remote computing system and the trained vessel ID model 310 may be loaded/installed onto venipuncture devices 100. The training process 1100a receives a training corpus of ultrasound image sequence sets 1110. Each ultrasound image sequence set 1110 in the training corpus includes a corresponding sequence of ultrasound image frames 1120, 1120a-n of an anatomy portion of a subject captured by a corresponding ultrasound image device as the corresponding ultrasound image device scans across the anatomy portion. The anatomy portion may include an arm of a human subject. As such, each corresponding sequence of ultrasound image frames 1120 may include anatomy portions of a pool of different subjects captured by ultrasound image devices. Each corresponding ultrasound image frame 1120 includes manual annotations that identify one or more corresponding ground-truth vessel locations 1122 in the corresponding ultrasound image frame 1120. Scenarios may exist where some of the ultrasound image frames 1120 may omit manual annotations when no vessel locations exist. Notably, each image frame 1120 may be represented by a plurality of pixels, thereby providing location information for each ground-truth vessel location 1122 identified by the manual annotations.

Moreover, each corresponding ultrasound image frame 1120 may be paired with three-dimensional positional data 1126 of the corresponding ultrasound image device when the corresponding ultrasound image frame 1120 was captured by the corresponding ultrasound image device. As discussed above, the three-dimensional positional data 1126 may be used to map the locations of vessels identified in two-dimensional image frames (i.e., via the vessel masks 312) into the three-dimensional space for constructing the three-dimensional vessel structure map 700.

With continued reference to FIG. 11A, for each ultrasound image sequence set 1110 in the training corpus, the vessel ID training process 1100a trains, using a deep neural network 1130, the vessel ID model 310 on the corresponding sequence of ultrasound image frames 1120 to teach the vessel ID model 310 to learn how to generate a corresponding predicted vessel mask 1132 for each corresponding ultrasound image frame 1120 that identifies the one or more corresponding ground-truth vessel locations 1122. A loss module 1140 computes training losses/loss terms 1142 based on the predicted vessel masks 1132 output by the deep neural network 1130 for each ultrasound image frame 1120 relative to the one or more corresponding ground-truth vessel locations 1122 identified by the manual annotations in the ultrasound image frame 1120. The vessel ID model training process 1100a may update parameters of the deep neural network based on the training losses/loss terms 1142 until parameters of the deep neural network 1130 converge to obtain the trained vessel ID model 310. The loss module 1140 may employ a cross-entropy loss function. Additionally, the loss module 1140 may counteract overfitting by applying L2-regularization.

Referring now to FIG. 11B, an example contact detection model training process 1100, 1100b is shown that may be used to train the contact detection model 320. The training process 1100b may execute on data processing hardware of a remote computing system and the contact detection model 320 may be loaded/installed onto venipuncture devices 100. Similar to the vessel ID model training process 1100a of FIG. 11A, the contact detection model training process 1100b receives the training corpus of ultrasound image sequence sets 1110. Here, for each respective ultrasound image frame 1120 from the training corpus of ultrasound image sequence sets 1110 that includes the presence of an insufficient acoustic interface, the respective ultrasound image frame further includes additional manual annotations that identify one or more corresponding ground-truth insufficient acoustic interface locations 1124 in the respective ultrasound image frame 1120. As used herein, each ground-truth insufficient acoustic interface location 1124 indicates a location where an insufficient acoustic interface exists between the ultrasound image device that captured the corresponding ultrasound image frame 1120 and the exterior of the anatomy portion. For instance, an area of an arm that bends opposite the elbow may create an insufficient acoustic interface when an ultrasound image device traverses across the skin at the area where the arm bends.

For each respective ultrasound image frame 1120 from the training corpus of ultrasound image sequence sets 1110 that includes the presence of the insufficient acoustic interface, the contact detection model training process 1100b trains, using a deep neural network 1130, 1130b, the contact detection model 320 on each respective ultrasound image frame 1120 to teach the contact detection model to learn how to generate a corresponding predicted contact detection mask 1134 for each respective ultrasound image frame 1120 that identifies the one or more corresponding ground-truth insufficient acoustic interface locations 1124. A loss module 1140 computes training losses/loss terms 1144 based on the predicted contact detection masks 1134 output by the deep neural network 1130b for each respective ultrasound image frame 1120 relative to the one or more corresponding ground-truth insufficient acoustic interface locations 1124 identified by the additional manual annotations in the respective ultrasound image frame 1120. The contact detection model training process 1100b may update parameters of the deep neural network 1130b based on the training losses/loss terms 1144 until parameters of the deep neural network 1130b converge to obtain the trained contact detection model 320. The loss module 1140 may employ a cross-entropy loss function. Additionally, the loss module 1140 may counteract overfitting by applying L2-regularization

Notably, the vessel ID model training process 1100a may use a first neural network 1130a to train the vessel ID model 310 while the contact detection model training process 1100b may use a second neural network 1130b different than the first neural network 1130a to train the contact detection model 320. As such, the vessel ID model 310 and the contact detection model 320 may be trained separately and include different neural network architectures.

Referring to FIG. 11C, in some implementations, the vessel ID model 310 and the contact detection model 320 are trained jointly by a joint training process 1100c. As with the training processes 1100a, 1100b of FIGS. 11A and 11B, the joint training process 1100c receives the training corpus of ultrasound image sequence sets 1110 whereby each corresponding ultrasound image frame 1120 includes manual annotations that identify the one or more corresponding ground-truth vessel locations 1122 in the corresponding ultrasound image frame 1120, the additional annotations that identify the one or more corresponding ground-truth insufficient acoustic interface locations 1124 (provided an insufficient acoustic interface exists in the image frame), and the three-dimensional positional data 1126 of the corresponding ultrasound image device when the corresponding ultrasound image frame 1120 was captured by the corresponding ultrasound image device. The joint training process 1100c uses the same deep neural network 1130 to train both the vessel ID model 310 and the contact detection model 320 on each corresponding sequence of ultrasound image frames 1120 to teach the vessel ID model 310 to learn how to generate the corresponding predicted vessel mask 1132 for each corresponding ultrasound image frame 1120 that identifies the one or more corresponding ground-truth vessel locations 1122 and the contact detection model 320 to learn how to generate the corresponding predicted contact detection mask 1134 for each respective ultrasound image frame 1120 that identifies the one or more corresponding ground-truth insufficient acoustic interface locations 1124.

In some implementations, the joint training process 1100c employs a first loss module 1140a that computes first training losses/loss terms 1142 and a second loss module 1140b that computes second training losses/loss terms 1144. The first loss module 1140a computes the first training losses/loss terms 1142 based on the predicted vessel masks 1132 output by the deep neural network 1130 for each ultrasound image frame 1120 relative to the one or more corresponding ground-truth vessel locations 1122 identified by the manual annotations in the ultrasound image frame 1120. Similarly, the second loss module 1140b computes the training losses/loss terms 1144 based on the predicted contact detection masks 1134 output by the deep neural network 1130 for each respective ultrasound image frame 1120 relative to the one or more corresponding ground-truth insufficient acoustic interface locations 1124 identified by the additional manual annotations in the respective ultrasound image frame 1120. The joint training process 1100c may update parameters of the deep neural network 1130 based on the first and second training losses/loss terms 1142, 1144 until parameters of the deep neural network 1130 converge to obtain a trained joint vessel ID and contact detection model 360. During inference, the trained joint vessel ID and contact detection model 360 may process an input ultrasound image frame and generate, as output, a corresponding vessel ID mask and a corresponding contact detection mask without requiring the use of two separate models to each process the same ultrasound image frames. As such, a joint model trained to predict both vessel ID masks and contact detection masks for a same input image frame reduces processing and memory costs, as well as latency, to improve overall performance.

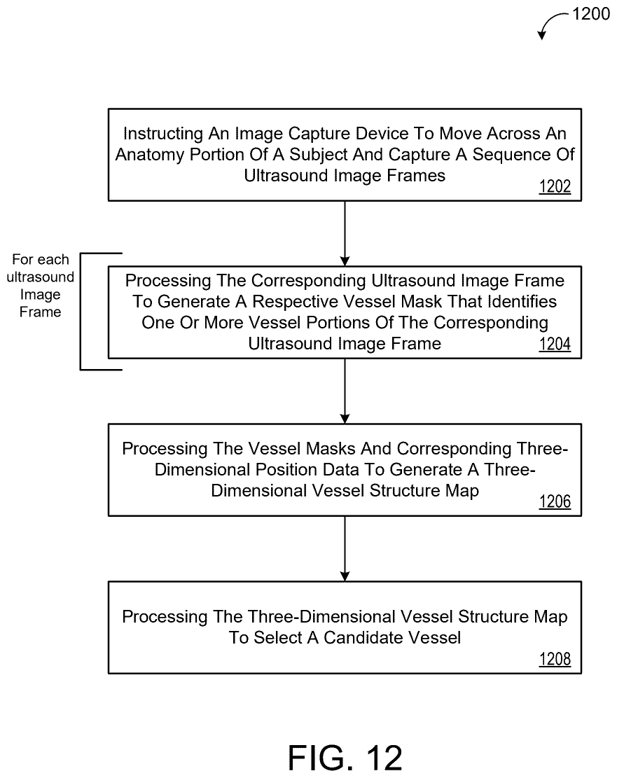

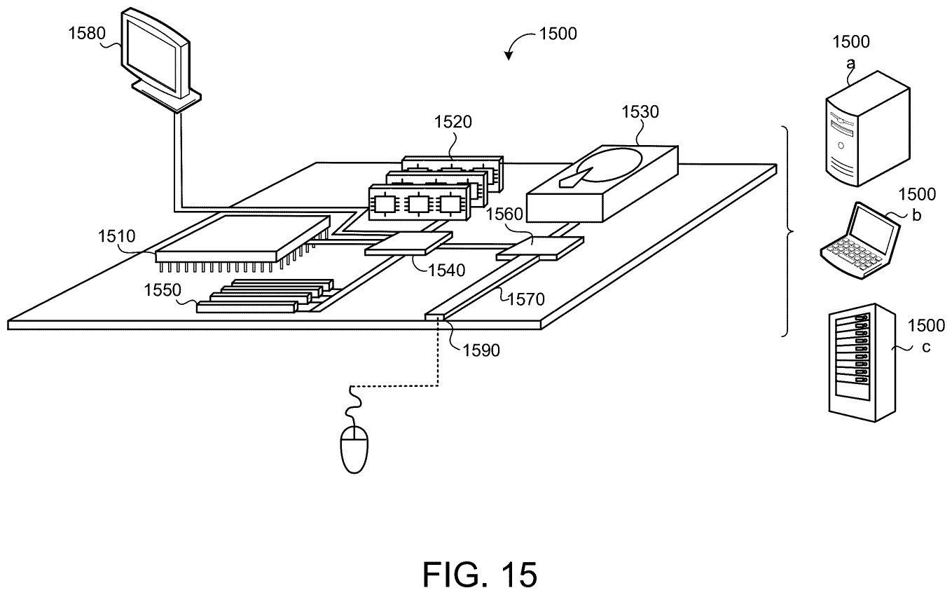

FIG. 12 is a flowchart of an example arrangement of operations for a computer-implemented method 1200 of performing site selection from a sequence of ultrasound image frames 154. The method 1200 may execute on the data processing hardware 1510 (FIG. 15) based on instructions stored on memory hardware 1520 (FIG. 15) in communication with the data processing hardware 1510. The data processing hardware 1510 and the memory hardware 1520 may reside on the remote system and/or on the venipuncture device 100 corresponding to a computing device 1500 (FIG. 15).

At operation 1202, the method 1200 includes instructing an ultrasonic device 150 to move across an anatomy portion of a subject and capture a sequence of ultrasound image frames 154 while the ultrasonic device 150 moves across the anatomy portion. At operation 1204, the method 1200 includes, for each corresponding ultrasound image frame 154 in the sequence of ultrasound image frames 154, processing the corresponding ultrasound image frame 154, using the vessel ID model 310, to generate a respective vessel mask 312 that identifies one or more vessel portions 314 of the corresponding ultrasound image frame 154. Each respective vessel portion 314 indicates where a respective vessel 156 is located in the corresponding ultrasound image frame 154.

At operation 1206, the method 1200 includes processing, using a vessel map generator 340, the vessel masks 312 generated for the sequence of ultrasound image frames 154 and corresponding three-dimensional position data 153 to generate a three-dimensional vessel structure map 700 representing vessels 156 within the anatomy portion of the subject. Here, each respective vessel mask 312 is paired with corresponding three-dimensional position data 153 of the ultrasonic device 150 when the ultrasonic device 150 captured the corresponding ultrasound image frame 154. At operation 1208, the method 1200 includes processing the three-dimensional vessel structure map 700 to select, from the vessels 156 represented in the three-dimensional vessel structure map 700, a candidate vessel 156C to target for venipuncture.

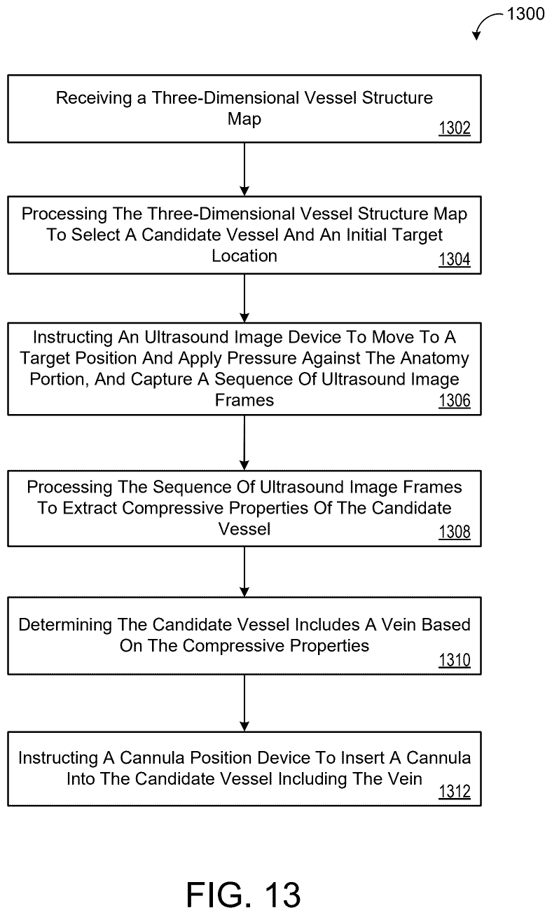

FIG. 13 is a flowchart of an example arrangement of operations for a computer-implemented method 1300 of performing vein confirmation from a sequence of ultrasound image frames 154. The method 1300 may execute on the data processing hardware 1510 (FIG. 15) based on instructions stored on the memory hardware 1520 (FIG. 15) in communication with the data processing hardware 1510. The data processing hardware 1510 and the memory hardware 1520 may reside on the remote system and/or on the venipuncture device 100 corresponding to the computing device 1500 (FIG. 15).

At operation 1302, the method 1300 includes receiving a three-dimensional vessel structure map 700 representing vessels 156 of an anatomy portion of a subject in a three-dimensional space. At operation 1304, the method 1300 includes, processing the three-dimensional vessel structure map 700 to select a candidate vessel 156C from the vessels 156 represented in the three-dimensional vessel structure map 700 to target for venipuncture and an initial target location 802 of the selected candidate vessel 156C. At operation 1306, the method 1300 includes instructing an ultrasound image device 150 to: move to a target position against the anatomy portion of the subject based on the initial target location 802 of the candidate vessel 156C; apply pressure against the anatomy portion to exert a force upon the candidate vessel 156C at the initial target location 802; and capture a sequence of ultrasound image frames 154 while the ultrasound image device 150 is applying the pressure against the anatomy portion of the subject from the target position.

At operation 1308, the method 1300 includes processing the sequence of ultrasound image frames 154 captured by the ultrasound image device 100 to extract compressive properties 412 of the candidate vessel 156C. At operation 1310, the method 1300 includes determining the candidate vessel 156C includes a vein based on the compressive properties 412 of the candidate vessel 156C. At operation 1312, the method 1300 includes instructing a cannula positioning device (i.e., cannula positioning mechanism) 134 to insert a cannula 130 into the candidate vessel 156C that includes the vein based on determining the candidate vessel 156C includes the vein.

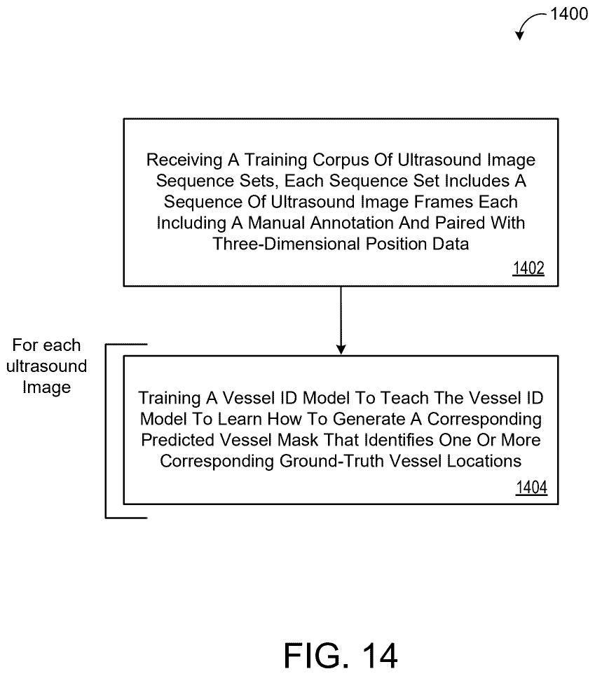

FIG. 14 is a flowchart of an example arrangement of operations for a computer-implemented method 1400 of training a vessel ID model 310. The method 1400 may execute on the data processing hardware 1510 (FIG. 15) based on instructions stored on the memory hardware 1520 (FIG. 15) in communication with the data processing hardware 1510. The data processing hardware 1510 and the memory hardware 1520 may reside on the remote system and/or on the venipuncture device 100 corresponding to a computing device 1500 (FIG. 15).

At operation 1402, the method 1400 includes receiving a training corpus of ultrasound image sequence sets 1110 with each ultrasound image sequence set 1110 including a corresponding sequence of ultrasound image frames 1120 of the anatomy portion captured by a corresponding ultrasound image device 150 as the corresponding ultrasound image device 150 scans across the anatomy portion of the subject. Here, each corresponding ultrasound image frame 1120 includes manual annotations that identify one or more corresponding ground-truth vessel locations 1122 in the corresponding ultrasound image frame 1120 and is paired with three-dimensional positional data 1126 of the corresponding ultrasound image device 150 when the corresponding ultrasound image frame 1120 was captured by the corresponding ultrasound image device 150. At operation 1404, for each ultrasound image sequence set 1110 in the training corpus, the method 1400 includes training a vessel ID model 310 on the corresponding sequence of ultrasound image frames 1120 to teach the vessel ID model 310 to learn how to generate a corresponding predicted vessel mask 1132 for each corresponding ultrasound image frame 1120 that identifies the one or more corresponding ground-truth vessel locations 1122.

FIG. 15 is a schematic view of an example computing device 1500 that may be used to implement the systems and methods described in this document. The computing device 1500 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1500 includes a processor 1510, memory 1520, a storage device 1530, a high-speed interface/controller 1540 connecting to the memory 1520 and high-speed expansion ports 1550, and a low speed interface/controller 1560 connecting to a low speed bus 1570 and a storage device 1530. Each of the components 1510, 1520, 1530, 1540, 1550, and 1560, are interconnected using various busses, and may be mounted on a common motherboard or in other manners as appropriate. The processor 1510 can process instructions for execution within the computing device 1500, including instructions stored in the memory 1520 or on the storage device 1530 to display graphical information for a graphical user interface (GUI) on an external input/output device, such as display 1580 coupled to high speed interface 1540. In other implementations, multiple processors and/or multiple buses may be used, as appropriate, along with multiple memories and types of memory. Also, multiple computing devices 1500 may be connected, with each device providing portions of the necessary operations (e.g., as a server bank, a group of blade servers, or a multi-processor system).

The memory 1520 stores information non-transitorily within the computing device 1500. The memory 1520 may be a computer-readable medium, a volatile memory unit(s), or non-volatile memory unit(s). The non-transitory memory 1520 may be physical devices used to store programs (e.g., sequences of instructions) or data (e.g., program state information) on a temporary or permanent basis for use by the computing device 1500. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 1530 is capable of providing mass storage for the computing device 1500. In some implementations, the storage device 1530 is a computer-readable medium. In various different implementations, the storage device 1530 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer-or machine-readable medium, such as the memory 1520, the storage device 1530, or memory on processor 1510.

The high speed controller 1540 manages bandwidth-intensive operations for the computing device 1500, while the low speed controller 1560 manages lower bandwidth-intensive operations. Such allocation of duties is exemplary only. In some implementations, the high-speed controller 1540 is coupled to the memory 1520, the display 1580 (e.g., through a graphics processor or accelerator), and to the high-speed expansion ports 1550, which may accept various expansion cards (not shown). In some implementations, the low-speed controller 1560 is coupled to the storage device 1530 and a low-speed expansion port 1590. The low-speed expansion port 1590, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1500a or multiple times in a group of such servers 1500a, as a laptop computer 1500b, or as part of a rack server system 1500c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
instructing an image capture device to:
move across an anatomy portion of a subject; and
capture a sequence of ultrasound image frames while the image capture device moves across the anatomy portion;
for each corresponding ultrasound image frame in the sequence of ultrasound image frames, processing, using a vessel identification model, the corresponding ultrasound image frame to generate a respective vessel mask that identifies one or more vessel portions of the corresponding ultrasound image frame, each respective vessel portion indicating where a respective vessel is located in the corresponding ultrasound image frame;
processing, using a vessel map generator, the vessel masks generated for the sequence of ultrasound image frames and corresponding three-dimensional position data to generate a three-dimensional vessel structure map representing vessels within the anatomy portion of the subject, each respective vessel mask paired with corresponding three-dimensional position data of the image capture device when the image capture device captured the corresponding ultrasound image frame; and
processing the three-dimensional vessel structure map to select, from the vessels represented in the three-dimensional vessel structure map, a candidate vessel to target for venipuncture.

2. The computer-implemented method of claim 1, wherein processing the three-dimensional vessel structure map to select the candidate vessel comprises:
processing the three-dimensional vessel structure map to identify a plurality of vessels within the anatomy portion of the subject;
from each corresponding vessel of the plurality of vessels identified, extracting respective vessel properties of the corresponding vessel;
ranking the plurality of vessels identified based on the respective vessel properties extracted for each of the plurality of vessels; and
selecting the highest rank vessel among the plurality of vessels as the candidate vessel to target for venipuncture.

3. The computer-implemented method of claim 2, wherein the respective vessel properties extracted from each corresponding vessel comprise at least one of a diameter of the corresponding vessel, an angle of the corresponding vessel relative to a reference angle, a depth of the corresponding vessel from an exterior surface of the anatomy portion, or any branch of vessels branching from the corresponding vessel.

4. The computer-implemented method of claim 1, wherein the vessel identification model comprises a deep neural network architecture.

5. The computer-implemented method of claim 1, wherein the operations further comprise:
for each corresponding ultrasound image frame in the sequence of ultrasound image frames:
processing, using a contact detection model, the corresponding ultrasound image frame to generate a respective contact mask identifying a presence of any insufficient acoustic interface portions of the corresponding ultrasound image frame that indicate where an insufficient acoustic interface is located in the corresponding ultrasound image frame;
comparing the respective vessel mask and the respective contact mask to determine whether the respective contact mask identified any insufficient acoustic interface portions that overlap with any of the vessel portions identified by the respective vessel mask in the corresponding ultrasound image frame; and
validating the respective vessel mask to discard any vessel portions identified by the respective vessel mask that overlap with insufficient acoustic interface portions identified by the respective contact mask,
wherein processing the vessel masks generated for the sequence of ultrasound image frames comprises processing, using the vessel map generator, the validated vessel masks and the corresponding three-dimensional position data to generate the three-dimensional vessel structure map.

6. The computer-implemented method of claim 5, wherein the insufficient acoustic interface indicates an insufficient acoustic interface between an ultrasound sensor of the image capture device and the anatomy portion of the subject.

7. The computer-implemented method of claim 5, wherein:
the vessel identification model comprises a first deep neural network architecture configured to receive, as input, the sequence of ultrasound image frames and to generate, as output, the vessel masks; and
the contact detection model comprises a second deep neural network architecture different from the first neural network and is configured to receive, as input, the sequence of ultrasound image frames and to generate, as output, the contact masks.

8. The computer-implemented method of claim 5, wherein the vessel identification model and the contact detection model each comprise a same deep neural network architecture configured to receive, as input, the sequence of ultrasound image frames and to generate, as output, both the vessel masks and the contact masks.

9. A computer-implemented method executed on data processing hardware that causes the data processing hardware to perform operations comprising:
receiving a three-dimensional vessel structure map representing vessels of an anatomy portion of a subject in a three-dimensional space;
processing the three-dimensional vessel structure map to select:
a candidate vessel from the vessels represented in the three-dimensional vessel structure map to target for venipuncture; and
an initial target location of the selected candidate vessel to puncture;
instructing an ultrasound image device to:
move to a target position against the anatomy portion of the subject based on the initial target location of the candidate vessel;
apply, from the target position against the anatomy portion of the subject, pressure against the anatomy portion to exert a force upon the candidate vessel at the initial target location; and
capture a sequence of ultrasound image frames while the ultrasound image devices is applying the pressure against the anatomy portion of the subject from the target position;
processing the sequence of ultrasound image frames captured by the ultrasound image device to extract compressive properties of the candidate vessel;
determining the candidate vessel comprises a vein based on the compressive properties of the candidate vessel; and
based on determining the candidate vessel comprises the vein, instructing a cannula positioning device to insert a cannula into the candidate vessel comprising the vein.

10. The computer-implemented method of claim 9, wherein instructing the ultrasound image device to move to the target position further comprises instructing the ultrasound image device to:
move to a target orientation that aligns a longitudinal axis of the ultrasound image in a direction substantially perpendicular to a longitudinal axis of the candidate vessel at the target location,
wherein instructing the ultrasound image device to apply pressure comprises instructing the ultrasound image device to apply, from the target position and the target orientation, the pressure against the anatomy portion to exert the force upon the candidate vessel in the direction substantially perpendicular to the longitudinal axis of the candidate vessel at the target location.

11. The computer-implemented method of claim 9, wherein instructing the ultrasound image device to apply pressure comprises instructing the ultrasound image device to increase pressure from an initial pressure value to a final pressure value during a predetermined duration of time.

12. The computer-implemented method of claim 9, determining the candidate vessel comprises a vein comprises executing a vein confirmation model configured to:
receive, as input, the compressive properties of the candidate vessel and a magnitude of the force exerted upon the candidate vessel at the target location; and
generate a classification output classifying the candidate vessel as the vein.

13. The computer-implemented method of claim 12, wherein the vein confirmation model is trained to:
classify vessels as a vein when the compressive properties of the vessels indicate a decreasing cross-sectional area responsive to increases in magnitude of force exerted upon the vessels; and
classify vessels as arteries when the compressive properties of the vessels indicate that the cross-sectional areas do not decrease responsive to increases in the magnitude of force.

14. The computer-implemented method of claim 9, wherein the operations further comprise:
based on determining the candidate vessel comprises the vein, instructing the cannula positioning device to orient a longitudinal axis of the cannula at a target angle relative to a longitudinal axis of the vein,
wherein instructing the cannula positioning device to insert the cannula into the candidate vessel comprising the vein comprises instructing the cannula positioning device to insert the cannula into the candidate vessel while the longitudinal axis of the cannula is oriented at the target angle relative to the longitudinal axis of the vein.

15. The computer-implemented method of claim 9, wherein processing the three-dimensional vessel structure map to select the candidate vessel comprises:
processing the three-dimensional vessel structure map to identify a plurality of vessels within the anatomy portion of the subject;
from each corresponding vessel of the plurality of vessels identified, extracting respective vessel properties of the corresponding vessel;
ranking the plurality of vessels identified based on the respective vessel properties extracted for each of the plurality of vessels; and
selecting the highest rank vessel among the plurality of vessels as the candidate vessel to target for venipuncture.

16. The computer-implemented method of claim 15, wherein the respective vessel properties extracted from each corresponding vessel comprise at least one of a diameter of the corresponding vessel, an angle of the corresponding vessel relative to a reference angle, a depth of the corresponding vessel from an exterior surface of the anatomy portion, or any branch vessels branching from the corresponding vessel.

17. The computer-implemented method of claim 9, wherein processing the sequence of ultrasound image frames captured by the ultrasound image device to extract compressive properties of the candidate vessel comprises:
for each ultrasound image frame in the sequence of ultrasound image frames:
processing, using a vessel identification model, the corresponding ultrasound image frame to generate a respective vessel mask that identifies a respective portion of the corresponding ultrasound image frame where the candidate vessel is located;
processing the respective vessel mask to determine a cross-sectional area of the candidate vessel; and
determining the compressive properties of the candidate vessel based on the cross-sectional areas of the candidate vessel determined for the sequence of ultrasound image frames.

18. The computer-implemented method of claim 9, wherein the sequence of ultrasound image frames comprise two-dimensional ultrasound image frames.

19. The computer-implemented method of claim 9, wherein the operations further comprise, after determining the candidate vessel comprises the vein:
instructing the image capture device to capture, from the target position against the anatomy portion of the subject, an additional ultrasound image frame; and
processing the additional ultrasound image frame to identify the candidate vessel and determine a final target location of the candidate vessel to puncture,
wherein instructing the cannula positioning device to insert the cannula into the candidate vessel comprises instructing the cannula positioning device to insert the cannula into the candidate vessel at the final target location.

20. A venipuncture device comprising:
data processing hardware; and
memory hardware in communication with the data processing hardware, the memory hardware storing instructions that when executed on the data processing hardware cause the data processing hardware to perform operations comprising:
instructing an image capture device to:
move across an anatomy portion of a subject; and
capture a sequence of ultrasound image frames while the image capture device moves across the anatomy portion;
for each corresponding ultrasound image frame in the sequence of ultrasound image frames, processing, using a vessel identification model, the corresponding ultrasound image frame to generate a respective vessel mask that identifies one or more vessel portions of the corresponding ultrasound image frame, each respective vessel portion indicating where a respective vessel is located in the corresponding ultrasound image frame;
processing, using a vessel map generator, the vessel masks generated for the sequence of ultrasound image frames and corresponding three-dimensional position data to generate a three-dimensional vessel structure map representing vessels within the anatomy portion of the subject, each respective vessel mask paired with corresponding three-dimensional position data of the image capture device when the image capture device captured the corresponding ultrasound image frame; and
processing the three-dimensional vessel structure map to select, from the vessels represented in the three-dimensional vessel structure map, a candidate vessel to target for venipuncture.

21. The venipuncture device of claim 20, wherein processing the three-dimensional vessel structure map to select the candidate vessel comprises:
processing the three-dimensional vessel structure map to identify a plurality of vessels within the anatomy portion of the subject;
from each corresponding vessel of the plurality of vessels identified, extracting respective vessel properties of the corresponding vessel;
ranking the plurality of vessels identified based on the respective vessel properties extracted for each of the plurality of vessels; and
selecting the highest rank vessel among the plurality of vessels as the candidate vessel to target for venipuncture.

22. The venipuncture device of claim 21, wherein the respective vessel properties extracted from each corresponding vessel comprise at least one of a diameter of the corresponding vessel, an angle of the corresponding vessel relative to a reference angle, a depth of the corresponding vessel from an exterior surface of the anatomy portion, or any branch of vessels branching from the corresponding vessel.

23. The venipuncture device of claim 20, wherein the vessel identification model comprises a deep neural network architecture.

24. The venipuncture device of claim 20, wherein the operations further comprise:
for each corresponding ultrasound image frame in the sequence of ultrasound image frames:
processing, using a contact detection model, the corresponding ultrasound image frame to generate a respective contact mask identifying a presence of any insufficient acoustic interface portions of the corresponding ultrasound image frame that indicate where an insufficient acoustic interface is located in the corresponding ultrasound image frame;
comparing the respective vessel mask and the respective contact mask to determine whether the respective contact mask identified any insufficient acoustic interface portions that overlap with any of the vessel portions identified by the respective vessel mask in the corresponding ultrasound image frame; and
validating the respective vessel mask to discard any vessel portions identified by the respective vessel mask that overlap with insufficient acoustic interface portions identified by the respective contact mask,
wherein processing the vessel masks generated for the sequence of ultrasound image frames comprises processing, using the vessel map generator, the validated vessel masks and the corresponding three-dimensional position data to generate the three-dimensional vessel structure map.

25. The venipuncture device of claim 24, wherein the insufficient acoustic interface indicates an insufficient acoustic interface between an ultrasound sensor of the image capture device and the anatomy portion of the subject.

26. The venipuncture device of claim 24, wherein:
the vessel identification model comprises a first deep neural network architecture configured to receive, as input, the sequence of ultrasound image frames and to generate, as output, the vessel masks; and
the contact detection model comprises a second deep neural network architecture different from the first neural network and is configured to receive, as input, the sequence of ultrasound image frames and to generate, as output, the contact masks.

27. The venipuncture device of claim 24, wherein the vessel identification model and the contact detection model each comprise a same deep neural network architecture configured to receive, as input, the sequence of ultrasound image frames and to generate, as output, both the vessel masks and the contact masks.
